(19) United States
(12) Patent Application Publication
(10) Pub. No.: US 2017/0057958 A1
(43) Pub. Date: Mar. 2, 2017

(54) ENANTIOMERS OF THE N-(2-AMINO-5-FLUORO-2-METHYLPENTYL)-8-[(2,6-DIFLUOROBENZYL)OXY]-2-METHYLIMIDAZO[1,2-A]PYRIDINE-3-CARBOXAMIDE, AS WELL AS OF THE DI- AND TRIFLUORO DERIVATIVES FOR THE TREATMENT OF CARDIOVASCULAR DISEASES

(71) Applicant: BAYER PHARMA AKTIENGESELLSCHAFT, Berlin, OT (DE)

(72) Inventors: Alexandros Vakalopoulos, Hilden (DE); Markus Follmann, Köln (DE); Johannes-Peter Stasch, Grottaferrate (RM) (IT); Damian Brockschnieder, Haan (DE); Frank Wunder, Wuppertal (DE); Tobias Marquardt, Wuppertal (DE); Lisa Dietz, Wuppertal (DE); Thomas Mondritzki, Velbert (DE); Dieter Lang, Velbert (DE); Volkhart Min-Jian Li, Velbert (DE)

(73) Assignee: BAYER PHARMA AKTIENGESELLSCHAFT, Berlin, OT (DE)

(21) Appl. No.: 15/308,279

(22) PCT Filed: Apr. 29, 2015
(86) PCT No.: PCT/EP2015/059274

§ 371 Date: Nov. 1, 2016

(30) Foreign Application Priority Data
May 2, 2014 (EP) 14166912.7

(51) Int. Cl. C07D 471/04; A61K 45/06; A61K 31/437

(52) U.S. Cl. CPC C07D 471/04; A61K 31/437; A61K 45/06

(57) ABSTRACT

The present application relates to novel 6-hydrogen-substituted imidazo[1,2-a]pyridine-3-carboxamides, to processes for preparation thereof, to the use thereof, alone or in combinations, for treatment and/or prophylaxis of diseases, and to the use thereof for production of medicaments for treatment and/or prophylaxis of diseases, especially for treatment and/or prophylaxis of cardiovascular disorders.

The present application relates to novel 6-hydrogen-substituted imidazo[1,2-a]pyridine-3-carboxamides, to processes for preparation thereof, to the use thereof, alone or in combinations, for treatment and/or prophylaxis of diseases, and to the use thereof for production of medicaments for treatment and/or prophylaxis of diseases, especially for treatment and/or prophylaxis of cardiovascular disorders.

One of the most important cellular transmission systems in mammalian cells is cyclic guanosine monophosphate (cGMP). Together with nitrogen monoxide (NO), which is released from the endothelium and transmits hormonal and mechanical signals, it forms the NO/cGMP system. Guanylate cyclases catalyse the biosynthesis of cGMP from guanosine triphosphate (GTP). The representatives of this family known to date can be classified into two groups either by structural features or by the type of ligands: the particulate guanylate cyclases which can be stimulated by natriuretic peptides, and the soluble guanylate cyclases which can be stimulated by NO. The soluble guanylate cyclases consist of two subunits and very probably contain one haem per heterodimer, which is part of the regulatory centre. This is of central importance for the activation mechanism. NO is able to bind to the iron atom of haem and thus markedly increase the activity of the enzyme. Haem-free preparations cannot, by contrast, be stimulated by NO. Carbon monoxide (CO) is also able to bind to the central iron atom of haem, but the stimulation by CO is much less than that by NO.

By forming cGMP, and owing to the resulting regulation of phosphodiesterases, ion channels and protein kinases, guanylate cyclase plays an important role in various physiological processes, in particular in the relaxation and proliferation of smooth muscle cells, in platelet aggregation and platelet adhesion and in neuronal signal transmission, and also in disorders which are based on a disruption of the aforementioned processes. Under pathophysiological conditions, the NO/cGMP system can be suppressed, which can lead, for example, to hypertension, platelet activation, increased cell proliferation, endothelial dysfunction, atherosclerosis, angina pectoris, heart failure, myocardial infarction, thromboses, stroke and sexual dysfunction.

Owing to the expected high efficiency and low level of side effects, a possible NO-independent treatment for such disorders by targeting the influence of the cGMP signal pathway in organisms is a promising approach.

Hitherto, for the therapeutic stimulation of the soluble guanylate cyclase, use has exclusively been made of compounds such as organic nitrates whose effect is based on NO. The latter is formed by bioconversion and activates soluble guanylate cyclase by attacking the central iron atom of haem. In addition to the side effects, the development of tolerance is one of the crucial disadvantages of this mode of treatment.

In recent years, some substances have been described which stimulate soluble guanylate cyclase directly, i.e. without prior release of NO, such as, for example, 3-(5′-hydroxymethyl-2′-furyl)-1-benzylindazole [YC-1; Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

Various imidazo[1,2-a]pyridine derivatives which can be used for treating disorders are described, inter alia, in EP 0 266 890-A1, WO 89/03833-A1, JP 01258674-A [cf. Chem. Abstr. 112:178986], WO 96/34866-A1, EP 1 277 754-A1, WO 2006/015737-A1, WO 2008/008539-A2, WO 2008/082490-A2, WO 2008/134553-A1, WO 2010/030538-A2, WO 2011/113606-A1 and WO 2012/165399-A1.

It was an object of the present invention to provide novel substances which act as stimulators of soluble guanylate cyclase and are suitable as such for treatment and/or prophylaxis of diseases.

The present invention provides compounds selected from the group consisting of

ent-N-(2-amino-5-fluoro-2-methylpentyl)-8-[(2,6-difluorobenzyl)oxy]-2-methylimidazo[1,2-a]pyridine-3-carboxamide (enantiomer A)

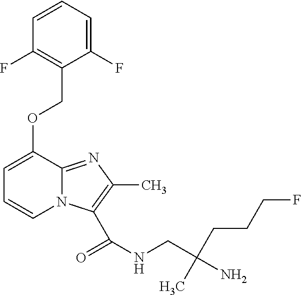 [SMILES: Cc1nc2c(OCc3c(F)cccc3F)cccn2c1C(=O)NCC(C)(N)CCCF]

and

ent-N-(2-amino-5-fluoro-2-methylpentyl)-8-[(2,6-difluorobenzyl)oxy]-2-methylimidazo[1,2-a]pyridine-3-carboxamide (enantiomer B)

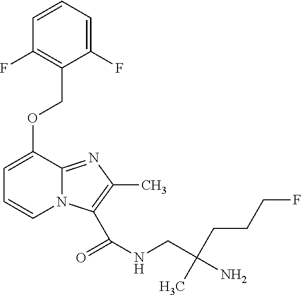 [SMILES: Cc1nc2c(OCc3c(F)cccc3F)cccn2c1C(=O)NCC(C)(N)CCCF]

and

ent-N-(2-amino-4,4-difluoro-2-methylbutyl)-8-[(2,6-difluorobenzyl)oxy]-2-methylimidazo[1,2-a]pyridine-3-carboxamide (enantiomer A)

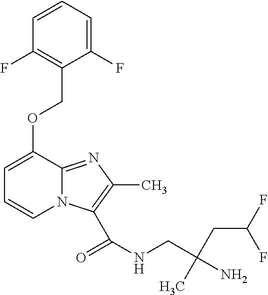 [SMILES: Cc1nc2c(OCc3c(F)cccc3F)cccn2c1C(=O)NCC(C)(N)CC(F)F]

and

ent-N-(2-amino-4,4-difluoro-2-methylbutyl)-8-[(2,6-difluorobenzyl)oxy]-2-methylimidazo[1,2-a]pyridine-3-carboxamide (enantiomer B)

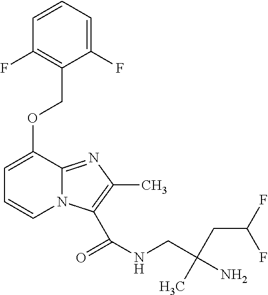 [SMILES: Cc1nc2c(OCc3c(F)cccc3F)cccn2c1C(=O)NCC(C)(N)CC(F)F]

and

ent-N-(2-amino-5,5,5-trifluoro-2-methylpentyl)-8-[(2,6-difluorobenzyl)oxy]-2-methylimidazo[1,2-a]pyridine-3-carboxamide (enantiomer A)

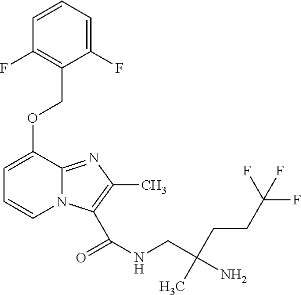 [SMILES: Cc1nc2c(OCc3c(F)cccc3F)cccn2c1C(=O)NCC(C)(N)CCC(F)(F)F]

and

ent-N-(2-amino-5,5,5-trifluoro-2-methylpentyl)-8-[(2,6-difluorobenzyl)oxy]-2-methylimidazo[1,2-a]pyridine-3-carboxamide (enantiomer B)

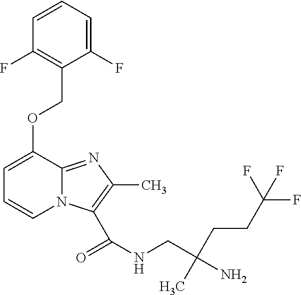 [SMILES: Cc1nc2c(OCc3c(F)cccc3F)cccn2c1C(=O)NCC(C)(N)CCC(F)(F)F]

and

ent-N-(2-amino-5,5,5-trifluoro-2-methylpentyl)-2-methyl-8-[(2,3,6-trifluorobenzyl)oxy]imidazo[1,2-a]pyridine-3-carboxamide (enantiomer B)

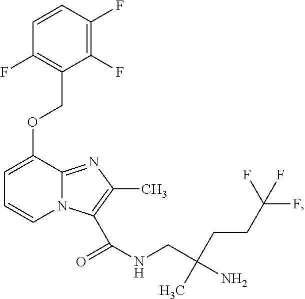 [SMILES: Cc1nc2c(OCc3c(F)ccc(F)c3F)cccn2c1C(=O)NCC(C)(N)CCC(F)(F)F]

and the N-oxides, salts, solvates, salts of the N-oxides and solvates of the N-oxides and salts thereof.

Preferred salts in the context of the present invention are physiologically acceptable salts of the compounds of the invention. Also encompassed are salts which are not themselves suitable for pharmaceutical applications but can be used, for example, for isolation or purification of the compounds according to the invention.

Physiologically acceptable salts of the compounds of the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds according to the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated from such mixtures of enantiomers and/or diastereomers in a known manner; chromatographic processes are preferably used for this purpose, especially HPLC chromatography on an achiral or chiral phase.

If the compounds according to the invention can occur in tautomeric forms, the present invention encompasses all the tautomeric forms.

The present invention also encompasses all suitable isotopic variants of the compounds according to the invention. An isotopic variant of a compound according to the invention is understood here to mean a compound in which at least one atom within the compound according to the invention has been exchanged for another atom of the same atomic number, but with a different atomic mass from the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to the comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting materials.

The present invention additionally also encompasses prodrugs of the compounds according to the invention. The term “prodrugs” in this context refers to compounds which may themselves be biologically active or inactive but are reacted (for example metabolically or hydrolytically) to give compounds according to the invention during their residence time in the body.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

Preference in the context of the present invention is given to the compound having the systematic name ent-N-(2-amino-5-fluoro-2-methylpentyl)-8-[(2,6-difluorobenzyl)oxy]-2-methylimidazo[1,2-a]pyridine-3-carboxamide (enantiomer A) and the structural formula

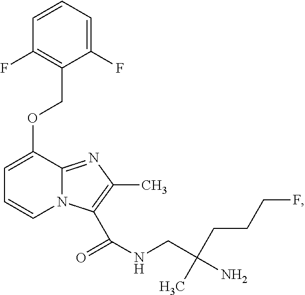 [SMILES: Cc1nc2c(OCc3c(F)cccc3F)cccn2c1C(=O)NCC(C)(N)CCCF]

and the N-oxides, salts, solvates, salts of the N-oxides and solvates of the N-oxides and salts thereof.

Preference in the context of the present invention is given to the compound having the systematic name ent-N-(2-amino-5-fluoro-2-methylpentyl)-8-[(2,6-difluorobenzyl)oxy]-2-methylimidazo[1,2-a]pyridine-3-carboxamide (enantiomer B) and the structural formula

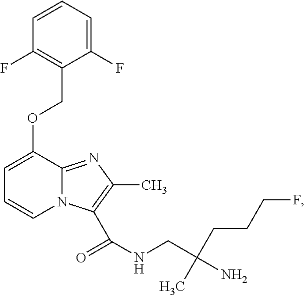 [SMILES: Cc1nc2c(OCc3c(F)cccc3F)cccn2c1C(=O)NCC(C)(N)CCCF]

and the N-oxides, salts, solvates, salts of the N-oxides and solvates of the N-oxides and salts thereof.

Preference in the context of the present invention is given to the compound having the systematic name ent-N-(2-amino-4,4-difluoro-2-methylbutyl)-8-[(2,6-difluorobenzyl)oxy]-2-methylimidazo[1,2-a]pyridine-3-carboxamide (enantiomer A) and the structural formula

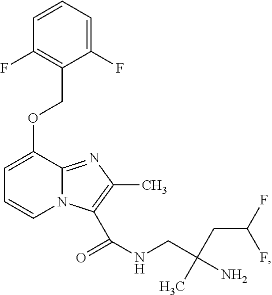 [SMILES: Cc1nc2c(OCc3c(F)cccc3F)cccn2c1C(=O)NCC(C)(N)CC(F)F]

and the N-oxides, salts, solvates, salts of the N-oxides and solvates of the N-oxides and salts thereof.

Preference in the context of the present invention is given to the compound having the systematic name ent-N-(2-amino-4,4-difluoro-2-methylbutyl)-8-[(2,6-difluorobenzyl)oxy]-2-methylimidazo[1,2-a]pyridine-3-carboxamide (enantiomer B) and the structural formula

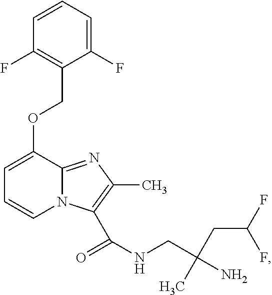 [SMILES: Cc1nc2c(OCc3c(F)cccc3F)cccn2c1C(=O)NCC(C)(N)CC(F)F]

and the N-oxides, salts, solvates, salts of the N-oxides and solvates of the N-oxides and salts thereof.

Preference in the context of the present invention is given to the compound having the systematic name ent-N-(2-amino-5,5,5-trifluoro-2-methylpentyl)-8-[(2,6-difluorobenzyl)oxy]-2-methylimidazo[1,2-a]pyridine-3-carboxamide (enantiomer A) and the structural formula

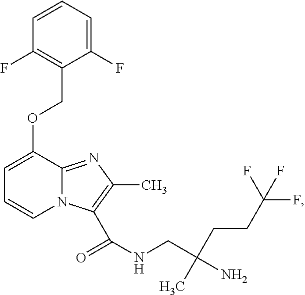 [SMILES: Cc1nc2c(OCc3c(F)cccc3F)cccn2c1C(=O)NCC(C)(N)CCC(F)(F)F]

and the N-oxides, salts, solvates, salts of the N-oxides and solvates of the N-oxides and salts thereof.

Preference in the context of the present invention is given to the compound having the systematic name ent-N-(2-amino-5,5,5-trifluoro-2-methylpentyl)-8-[(2,6-difluorobenzyl)oxy]-2-methylimidazo[1,2-a]pyridine-3-carboxamide (enantiomer B) and the structural formula

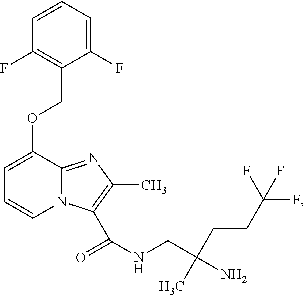 [SMILES: Cc1nc2c(OCc3c(F)cccc3F)cccn2c1C(=O)NCC(C)(N)CCC(F)(F)F]

and the N-oxides, salts, solvates, salts of the N-oxides and solvates of the N-oxides and salts thereof.

Preference in the context of the present invention is given to the compound having the systematic name ent-N-(2-amino-5,5,5-trifluoro-2-methylpentyl)-2-methyl-8-[(2,3,6-trifluorobenzyl)oxy]imidazo[1,2-a]pyridine-3-carboxamide (enantiomer B) and the structural formula

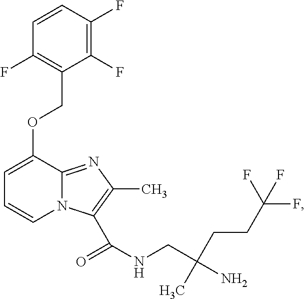 [SMILES: Cc1nc2c(OCc3c(F)ccc(F)c3F)cccn2c1C(=O)NCC(C)(N)CCC(F)(F)F]

and the N-oxides, salts, solvates, salts of the N-oxides and solvates of the N-oxides and salts thereof.

The invention further provides a process for preparing the inventive compounds, characterized in that

[A] a compound of the formula (I)

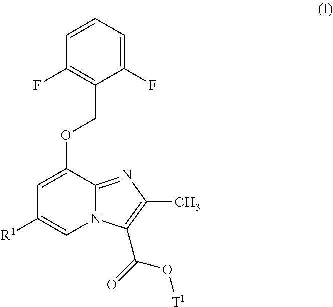

in which

R¹ represents hydrogen or chlorine,

T¹ represents (C₁-C₄)-alkyl or benzyl,

is reacted in an inert solvent in the presence of a suitable base or acid to give a carboxylic acid of the formula (II)

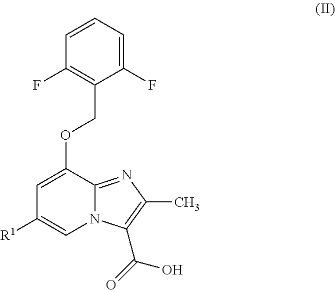

in which

R¹ represents hydrogen or chlorine,

and this is subsequently reacted in an inert solvent under amide coupling conditions with an amine selected from the group consisting of

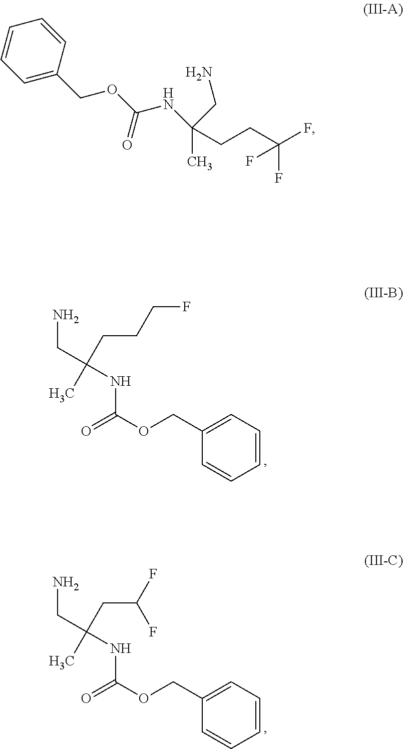 [SMILES: CC(CN)(CCC(F)(F)F)NC(=O)OCc1ccccc1 ; CC(CN)(CCCF)NC(=O)OCc1ccccc1 ; CC(CN)(CC(F)F)NC(=O)OCc1ccccc1]

to give compounds of the formula (IV)

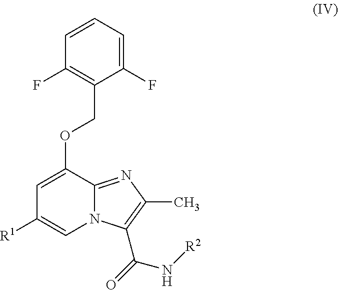

in which

R¹ represents hydrogen or chlorine,

and

R² represents (IV-A), (IV-B) or (IV-C)

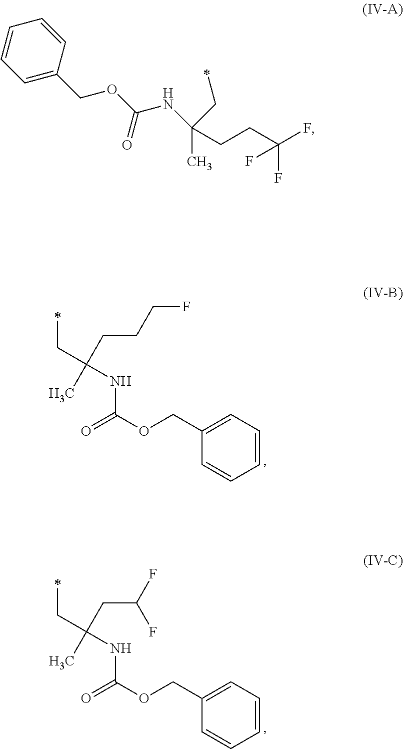

where * represents the point of attachment to the nitrogen atom,

and, if R¹ represents chlorine,

hydrogenating these in an inert solvent in the presence of a suitable transition metal catalyst,

and the resulting compounds are optionally converted with the appropriate (i) solvents and/or (ii) acids or bases into their solvates, salts and/or solvates of the salts.

The preparation processes described can be illustrated by way of example by the following synthesis scheme (Scheme 1):

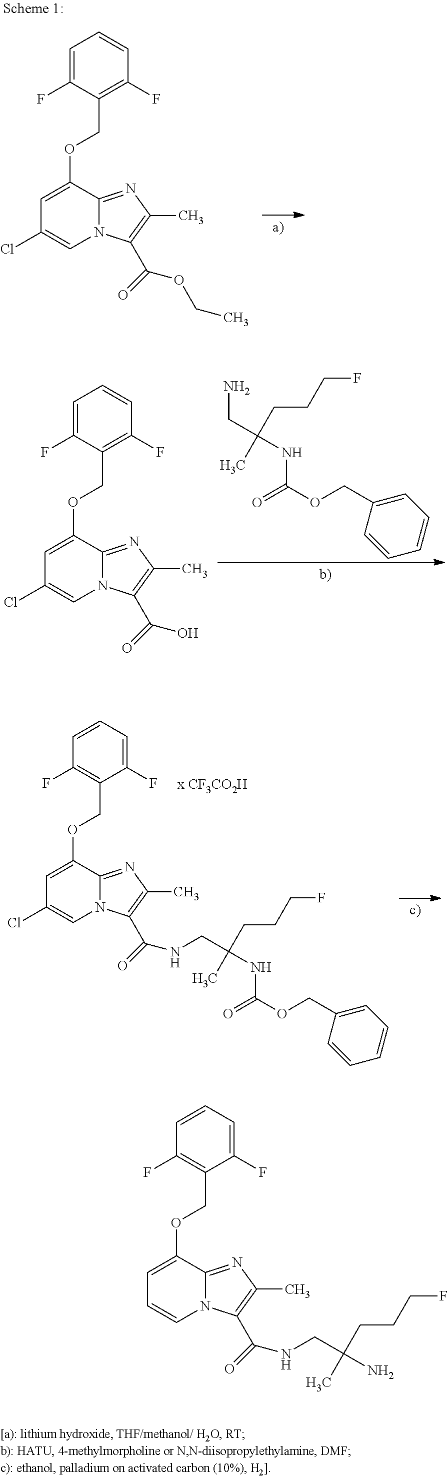 [SMILES: CCOC(=O)c1c(C)nc2c(OCc3c(F)cccc3F)cc(Cl)cn12 ; Cc1nc2c(OCc3c(F)cccc3F)cc(Cl)cn2c1C(=O)O ; CC(CN)(CCCF)NC(=O)OCc1ccccc1 ; Cc1nc2c(OCc3c(F)cccc3F)cc(Cl)cn2c1C(=O)NCC(C)(CCCF)NC(=O)OCc1ccccc1 ; Cc1nc2c(OCc3c(F)cccc3F)cccn2c1C(=O)NCC(C)(N)CCCF]

The compounds of the formulae (III-A), (III-B) and (III-C) are commercially available or known from the literature, or can be prepared in analogy to literature processes.

Suitable inert solvents for the amide coupling are, for example, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, halohydrocarbons such as dichloromethane, trichloromethane, tetrachloromethane, 1,2-dichloroethane, trichloroethylene or chlorobenzene, or other solvents such as acetone, ethyl acetate, acetonitrile, pyridine, dimethyl sulphoxide, N,N-dimethylformamide, N,N-dimethylacetamide, N,N′-dimethylpropyleneurea (DMPU) or N-methylpyrrolidone (NMP). It is likewise possible to use mixtures of the solvents mentioned. Preference is given to dichloromethane, tetrahydrofuran, dimethylformamide or mixtures of these solvents.

Suitable for use as condensing agents for the amide formation are, for example, carbodiimides such as N,N′-diethyl-, N,N′-dipropyl-, N,N′-diisopropyl-, N,N′-dicyclohexylcarbodiimide (DCC) or N-(3-dimethylaminopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium 3-sulphate or 2-tert-butyl-5-methylisoxazolium perchlorate, acylamino compounds such as 2-ethoxy-1-ethoxycarbonyl-1,2-dihydroquinoline or isobutyl chloroformate, propanephosphonic anhydride (T3P), 1-chloro-N,N,2-trimethylprop1-ene-1-amine, diethyl cyanophosphonate, bis(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), optionally in combination with further auxiliaries such as 1-hydroxybenzotriazole (HOBt) or N-hydroxysuccinimide (HOSu), and also as bases alkali metal carbonates, for example sodium carbonate or potassium carbonate or sodium bicarbonate or potassium bicarbonate, or organic bases such as trialkylamines, for example triethylamine, N-methylmorpholine, N-methylpiperidine or N,N-diisopropylethylamine. Preference is given to using TBTU in combination with N-methylmorpholine, HATU in combination with N,N-diisopropylethylamine or 1-chloro-N,N,2-trimethylprop-1-en-1-amine.

The condensation is generally carried out in a temperature range of from −20° C. to +100° C., preferably at from 0° C. to +60° C. The conversion can be carried out under atmospheric, elevated or reduced pressure (for example from 0.5 to 5 bar). In general, the reaction is carried out at atmospheric pressure.

Alternatively, the carboxylic acid of the formula (II) can also first be converted to the corresponding carbonyl chloride and the latter can then be converted directly or in a separate reaction with an amine of the formula (III) to the compounds of the invention. The formation of carbonyl chlorides from carboxylic acids is carried out by the methods known to those skilled in the art, for example by treatment with thionyl chloride, sulphuryl chloride or oxalyl chloride, in the presence of a suitable base, for example in the presence of pyridine, and optionally with addition of dimethylformamide, optionally in a suitable inert solvent.

The hydrolysis of the ester group T¹ in the compounds of the formula (I) is carried out by customary methods, by treating the esters in inert solvents with acids or bases, in which latter case the salts formed at first are converted to the free carboxylic acids by treating with acid. In the case of the tert-butyl esters, the ester hydrolysis is preferably carried out with acids. In the case of the benzyl esters, the ester hydrolysis is preferably carried out by hydrogenolysis with palladium on activated carbon or Raney nickel. Suitable inert solvents for this reaction are water or the organic solvents customary for ester hydrolysis. These preferably include alcohols such as methanol, ethanol, n-propanol, isopropanol, n-butanol or tert-butanol, or ethers such as diethyl ether, tetrahydrofuran, 2-methyltetrahydrofuran, dioxane or glycol dimethyl ether, or other solvents such as acetone, dichloromethane, dimethylformamide or dimethyl sulphoxide. It is also possible to use mixtures of the solvents mentioned. In the case of a basic ester hydrolysis, preference is given to using mixtures of water with dioxane, tetrahydrofuran, methanol and/or ethanol.

Suitable bases for the ester hydrolysis are the customary inorganic bases. These preferably include alkali metal or alkaline earth metal hydroxides, for example sodium hydroxide, lithium hydroxide, potassium hydroxide or barium hydroxide, or alkali metal or alkaline earth metal carbonates, such as sodium carbonate, potassium carbonate or calcium carbonate. Particular preference is given to sodium hydroxide or lithium hydroxide.

Suitable acids for the ester cleavage are generally sulphuric acid, hydrogen chloride/hydrochloric acid, hydrogen bromide/hydrobromic acid, phosphoric acid, acetic acid, trifluoroacetic acid, toluenesulphonic acid, methanesulphonic acid or trifluoromethanesulphonic acid, or mixtures thereof, optionally with addition of water. Preference is given to hydrogen chloride or trifluoroacetic acid in the case of the tert-butyl esters and to hydrochloric acid in the case of the methyl esters. The ester hydrolysis is generally carried out within a temperature range from 0° C. to +100° C., preferably at +0° C. to +50° C.

These conversions can be performed at atmospheric, elevated or reduced pressure (for example from 0.5 to 5 bar). In general, the reactions are in each case carried out at atmospheric pressure.

The amino protecting group used is preferably tert-butoxycarbonyl (Boc) or benzyloxycarbonyl (Z). Protecting groups used for a hydroxyl or carboxyl function are preferably tert-butyl or benzyl. These protecting groups are detached by customary methods, preferably by reaction with a strong acid such as hydrogen chloride, hydrogen bromide or trifluoroacetic acid in an inert solvent such as dioxane, diethyl ether, dichloromethane or acetic acid; it is optionally also possible to effect the detachment without an additional inert solvent. In the case of benzyl and benzyloxycarbonyl as protecting groups, these may also be removed by hydrogenolysis in the presence of a palladium catalyst. The detachment of the protecting groups mentioned can optionally be undertaken simultaneously in a one-pot reaction or in separate reaction steps.

The removal of the benzyl group is carried out here by customary methods known from protecting group chemistry, preferably by hydrogenolysis in the presence of a palladium catalyst, for example palladium on activated carbon, in an inert solvent, for example ethanol or ethyl acetate [see also, for example, T. W. Greene and P. G. M. Wuts, Protective Groups in Organic Synthesis, Wiley, New York, 1999].

The compounds of the formula (I) are known from the literature or can be prepared by reacting a compound of the formula (V)

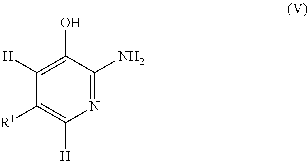

in which

R¹ represents hydrogen or chlorine,

in an inert solvent in the presence of a suitable base with a compound of the formula (VI)

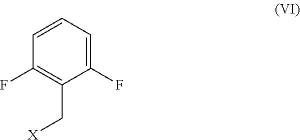

in which
X¹ represents a suitable leaving group, in particular chlorine, bromine, iodine, mesylate, triflate or tosylate,

to give a compound of the formula (VII)

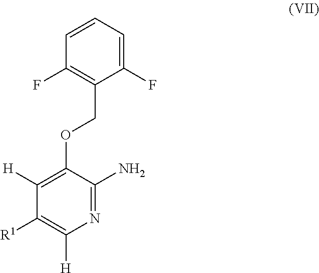

in which

R¹ represents hydrogen or chlorine,

and then reacting the latter in an inert solvent with a compound of the formula (VIII)

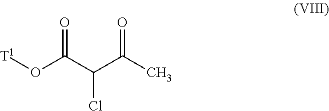

in which T¹ has the meanings given above.

The process described is illustrated in an exemplary manner by the scheme below (Scheme 2):

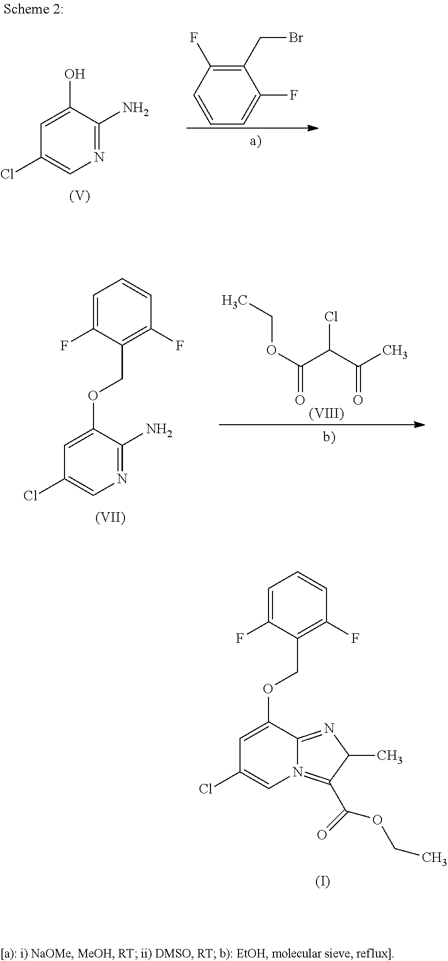

The synthesis sequence shown can be modified such that the respective reaction steps are carried out in a different order. An example of such a modified synthesis sequence is shown in Scheme 3.

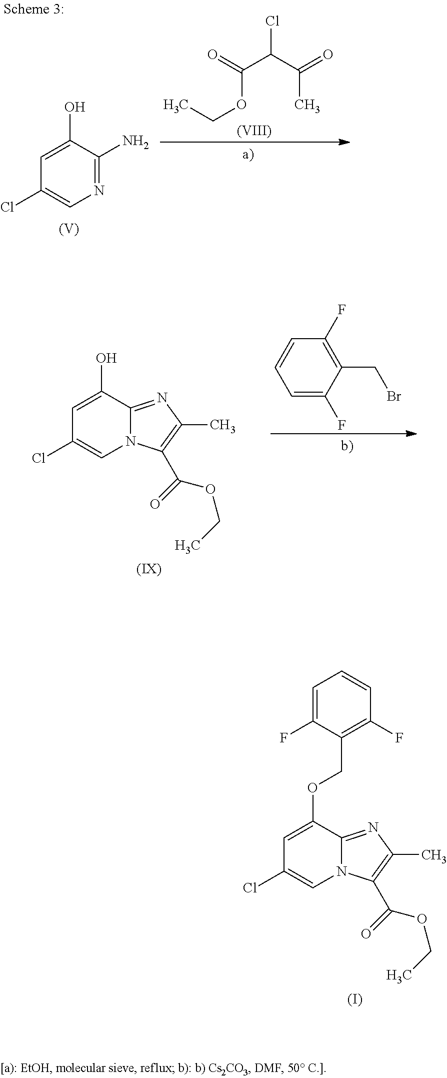

Inert solvents for the ring closure to give the imidazo[1,2-a]pyridine base skeleton (VII)+(VIII)→(I) or (V)+(VIII)→(IX) are the customary organic solvents. These preferably include alcohols such as methanol, ethanol, n-propanol, isopropanol, n-butanol, n-pentanol or tert-butanol, or ethers such as diethyl ether, tetrahydrofuran, 2-methyltetrahydrofuran, dioxane or glycol dimethyl ether, or other solvents such as acetone, dichloromethane, 1,2-dichloroethane, acetonitrile, dimethylformamide or dimethyl sulphoxide. It is also possible to use mixtures of the solvents mentioned. Preference is given to using ethanol.

The ring closure is generally carried out within a temperature range from +50° C. to +150° C., preferably at +50° C. to +100° C., optionally in a microwave.

The ring closure (VII)+(VIII)→(I) or (V)+(VIII)→(IX) is optionally carried out in the presence of dehydrating reaction additives, for example in the presence of molecular sieve (pore size 4 Å) or by means of a water separator. The reaction (VII)+(VIII)→(I) or (V)+(VIII)→(IX) is carried out using an excess of the reagent of the formula (VIII), for example with 1 to 20 equivalents of the reagent (VIII), optionally with addition of bases (for example sodium bicarbonate), in which case this addition can be carried out all at once or in several portions.

As an alternative to the introductions of the 2,6-difluorobenzyl group shown in Schemes 1 to 3, it is likewise possible—as shown in Scheme 4—to react these intermediates with alcohols of the formula (X) under conditions of the Mitsunobu reaction.

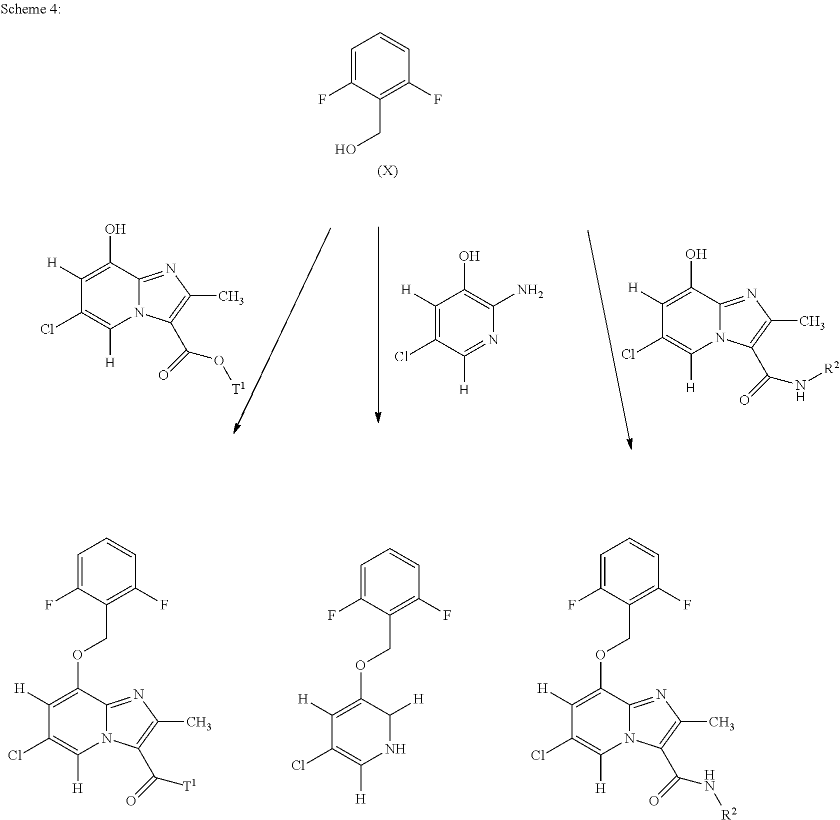

where

R² represents the compounds of the formulae (III-A), (III-B) and (III-C)

and

in which T¹ has the meanings given above.

Typical reaction conditions for such Mitsunobu condensations of phenols with alcohols can be found in the relevant literature, e.g. Hughes, D. L. Org. React. 1992, 42, 335; Dembinski, R. Eur. J. Org. Chem. 2004, 2763. Typically, the reaction is carried out using an activating agent, e.g. diethyl azodicarboxylate (DEAD) or diisopropyl azodicarboxylate (DIAD), and a phosphine reagent, e.g. triphenylphosphine or tributylphosphine, in an inert solvent, e.g. THF, dichloromethane, toluene or DMF, at a temperature between 0° C. and the boiling point of the solvent employed.

The compounds of the invention have valuable pharmacological properties and can be used for prevention and treatment of diseases in humans and animals. The compounds of the invention offer a further treatment alternative and thus enlarge the field of pharmacy.

The compounds of the invention bring about vasorelaxation and inhibition of platelet aggregation, and lead to a decrease in blood pressure and to a rise in coronary blood flow. These effects are mediated by a direct stimulation of soluble guanylate cyclase and an intracellular rise in cGMP. In addition, the compounds of the invention enhance the action of substances which increase the cGMP level, for example EDRF (endothelium-derived relaxing factor), NO donors, protoporphyrin IX, arachidonic acid or phenylhydrazine derivatives.

The compounds of the invention are suitable for treatment and/or prophylaxis of cardiovascular, pulmonary, thromboembolic and fibrotic disorders.

Accordingly, the compounds according to the invention can be used in medicaments for the treatment and/or prophylaxis of cardiovascular disorders such as, for example, high blood pressure (hypertension), resistant hypertension, acute and chronic heart failure, coronary heart disease, stable and unstable angina pectoris, peripheral and cardiac vascular disorders, arrhythmias, atrial and ventricular arrhythmias and impaired conduction such as, for example, atrioventricular blocks degrees I-III (AB block supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for the treatment and/or prophylaxis of thromboembolic disorders and ischaemias such as myocardial ischaemia, myocardial infarction, stroke, cardiac hypertrophy, transient and ischaemic attacks, preeclampsia, inflammatory cardiovascular disorders, spasms of the coronary arteries and peripheral arteries, oedema formation such as, for example, pulmonary oedema, cerebral oedema, renal oedema or oedema caused by heart failure, peripheral circulatory disturbances, reperfusion damage, arterial and venous thromboses, microalbuminuria, myocardial insufficiency, endothelial dysfunction, to prevent restenoses, for example after thrombolysis therapies, percutaneous transluminal angioplasties (PTA), transluminal coronary angioplasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for the treatment and/or prophylaxis of erectile dysfunction and female sexual dysfunction.

In the context of the present invention, the term “heart failure” encompasses both acute and chronic forms of heart failure, and also more specific or related types of disease, such as acute decompensated heart failure, right heart failure, left heart failure, global failure, ischaemic cardiomyopathy, dilated cardiomyopathy, hypertrophic cardiomyopathy, idiopathic cardiomyopathy, congenital heart defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic heart failure and systolic heart failure and acute phases of worsening of existing chronic heart failure (worsening heart failure).

In addition, the compounds of the invention can also be used for the treatment and/or prophylaxis of arteriosclerosis, impaired lipid metabolism, hypolipoproteinaemias, dyslipidaemias, hypertriglyceridaemias, hyperlipidaemias, hypercholesterolaemias, abetelipoproteinaemia, sitosterolaemia, xanthomatosis, Tangier disease, adiposity, obesity and of combined hyperlipidaemias and metabolic syndrome.

The compounds of the invention can also be used for the treatment and/or prophylaxis of primary and secondary Raynaud's phenomenon, microcirculation impairments, claudication, peripheral and autonomic neuropathies, diabetic microangiopathies, diabetic retinopathy, diabetic ulcers on the extremities, gangrene, CREST syndrome, erythematosis, onychomycosis, rheumatic disorders and for promoting wound healing.

The compounds according to the invention are furthermore suitable for treating urological disorders such as, for example, benign prostate syndrome (BPS), benign prostate hyperplasia (BPH), benign prostate enlargement (BPE), bladder outlet obstruction (BOO), lower urinary tract syndromes (LUTS, including Feline Urological Syndrome (FUS)), disorders of the urogenital system including neurogenic over-active bladder (OAB) and (IC), incontinence (UI) such as, for example, mixed urinary incontinence, urge urinary incontinence, stress urinary incontinence or overflow urinary incontinence (MUI, UUI, SUI, OUI), pelvic pain, benign and malignant disorders of the organs of the male and female urogenital system.

The compounds of the invention are also suitable for the treatment and/or prophylaxis of kidney disorders, in particular of acute and chronic renal insufficiency and acute and chronic renal failure. In the context of the present invention, the term “renal insufficiency” encompasses both acute and chronic manifestations of renal insufficiency, and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphataemia and/or need for dialysis. The present invention also encompasses the use of the compounds of the invention for the treatment and/or prophylaxis of sequelae of renal insufficiency, for example pulmonary oedema, heart failure, uraemia, anaemia, electrolyte disorders (for example hyperkalaemia, hyponatraemia) and disorders in bone and carbohydrate metabolism.

In addition, the compounds of the invention are also suitable for the treatment and/or prophylaxis of asthmatic disorders, pulmonary arterial hypertension (PAH) and other forms of pulmonary hypertension (PH) including left-heart disease-, HIV-, sickle cell anaemia-, thromboembolism (CTEPH)-, sarcoidosis-, COPD- or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

The compounds described in the present invention are also active ingredients for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post-stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, demyelinization, multiple sclerosis, thalamic degeneration, Creutzfeldt-Jakob dementia, HIV dementia, schizophrenia with dementia or Korsakoff's psychosis. They are also suitable for the treatment and/or prophylaxis of central nervous system disorders such as states of anxiety, tension and depression, CNS-related sexual dysfunctions and sleep disturbances, and for controlling pathological disturbances of the intake of food, stimulants and addictive substances.

In addition, the compounds of the invention are also suitable for controlling cerebral blood flow and are effective agents for controlling migraines. They are also suitable for the prophylaxis and control of sequelae of cerebral infarct (Apoplexia cerebri) such as stroke, cerebral ischaemias and skull-brain trauma. The compounds according to the invention can likewise be used for controlling states of pain and tinnitus.

In addition, the compounds of the invention have anti-inflammatory action and can therefore be used as anti-inflammatory agents for the treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin disorders and inflammatory eye disorders.

Furthermore, the compounds of the invention can also be used for the treatment and/or prophylaxis of autoimmune diseases.

The compounds of the invention are also suitable for the treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

The compounds of the invention are also suitable for controlling postoperative scarring, for example as a result of glaucoma operations.

The compounds of the invention can also be used cosmetically for ageing and keratinized skin.

Moreover, the compounds according to the invention are suitable for treatment and/or prophylaxis of hepatitis, neoplasms, osteoporosis, glaucoma and gastroparesis.

The present invention further provides for the use of the compounds according to the invention for treatment and/or prophylaxis of disorders, especially the disorders mentioned above.

The present invention further provides for the use of the compounds of the invention for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides the compounds of the invention for use in a method for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides for the use of the compounds of the invention for production of a medicament for treatment and/or prophylaxis of disorders, especially the aforementioned disorders.

The present invention further provides for the use of the compounds of the invention for production of a medicament for treatment and/or prophylaxis of heart failure, angina pectoris, hypertension, pulmonary hypertension, ischaemias, vascular disorders, renal insufficiency, thromboembolic disorders, fibrotic disorders and arteriosclerosis.

The present invention further provides a method for the treatment and/or prophylaxis of disorders, in particular the disorders mentioned above, using an effective amount of at least one of the compounds of the invention.

The present invention further provides a method for the treatment and/or prophylaxis of heart failure, angina pectoris, hypertension, pulmonary hypertension, ischaemias, vascular disorders, renal insufficiency, thromboembolic disorders, fibrotic disorders and arteriosclerosis using an effective amount of at least one of the compounds of the invention.

The compounds according to the invention can be used alone or, if required, in combination with other active compounds. The present invention further provides medicaments comprising at least one of the compounds of the invention and one or more further active compounds, especially for the treatment and/or prophylaxis of the aforementioned disorders. Preferred examples of active ingredients suitable for combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2 and/or 5, especially PDE 5 inhibitors such as sildenafil, vardenafil and tadalafil;
antithrombotic agents, by way of example and with preference from the group of the platelet aggregation inhibitors, the anticoagulants or the profibrinolytic substances;
hypotensive active compounds, by way of example and with preference from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics; and/or
active compounds altering lipid metabolism, for example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

Antithrombotic agents are preferably understood to mean compounds from the group of the platelet aggregation inhibitors, the anticoagulants or the profibrinolytic substances.

In a preferred embodiment of the invention, the compounds according to the invention are administered in combination with a platelet aggregation inhibitor, by way of example and with preference aspirin, clopidogrel, ticlopidine or dipyridamole.

In a preferred embodiment of the invention, the compounds of the invention are administered in combination with a thrombin inhibitor, by way of example and with preference ximelagatran, dabigatran, melagatran, bivalirudin or clexane.

In a preferred embodiment of the invention, the compounds according to the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the compounds according to the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds according to the invention are administered in combination with a calcium antagonist, by way of example and with preference nifedipine, amlodipine, verapamil or diltiazem.

In a preferred embodiment of the invention, the compounds according to the invention are administered in combination with an alpha-1-receptor blocker, by way of example and with preference prazosin.

In a preferred embodiment of the invention, the compounds according to the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an angiotensin AII antagonist, by way of example and with preference losartan, candesartan, valsartan, telmisartan or embursatan.

In a preferred embodiment of the invention, the compounds according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds according to the invention are administered in combination with an endothelin antagonist, by way of example and with preference bosentan, darusentan, ambrisentan or sitaxsentan.

In a preferred embodiment of the invention, the compounds according to the invention are administered in combination with a renin inhibitor, by way of example and with preference aliskiren, SPP-600 or SPP-800.

In a preferred embodiment of the invention, the compounds according to the invention are administered in combination with a mineralocorticoid receptor antagonist, by way of example and with preference spironolactone or eplerenone.

In a preferred embodiment of the invention, the compounds according to the invention are administered in combination with a loop diuretic, for example furosemide, torasemide, bumetanide and piretanide, with potassium-sparing diuretics, for example amiloride and triamterene, with aldosterone antagonists, for example spironolactone, potassium canrenoate and eplerenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the compounds according to the invention are administered in combination with a CETP inhibitor, by way of example and with preference dalcetrapib, BAY 60-5521, anacetrapib or CETP vaccine (CETi-1).

In a preferred embodiment of the invention, the compounds according to the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds according to the invention are administered in combination with a squalene synthesis inhibitor, by way of example and with preference BMS-188494 or TAK-475.

In a preferred embodiment of the invention, the compounds according to the invention are administered in combination with an ACAT inhibitor, by way of example and with preference avasimibe, melinamide, pactimibe, eflucimibe or SMP-797.

In a preferred embodiment of the invention, the compounds according to the invention are administered in combination with an MTP inhibitor, by way of example and with preference implitapide, BMS-201038, R-103757 or ITT-130.

In a preferred embodiment of the invention, the compounds according to the invention are administered in combination with a PPAR-gamma agonist, by way of example and with preference pioglitazone or rosiglitazone.

In a preferred embodiment of the invention, the compounds according to the invention are administered in combination with a PPAR-delta agonist, by way of example and with preference GW 501516 or BAY 68-5042.

In a preferred embodiment of the invention, the compounds according to the invention are administered in combination with a cholesterol absorption inhibitor, by way of example and with preference ezetimibe, tiqueside or pamaqueside.

In a preferred embodiment of the invention, the compounds according to the invention are administered in combination with a lipase inhibitor, by way of example and with preference orlistat.

In a preferred embodiment of the invention, the compounds according to the invention are administered in combination with a polymeric bile acid adsorber, by way of example and with preference cholestyramine, colestipol, colesolvam, CholestaGel or colestimide.

In a preferred embodiment of the invention, the compounds according to the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds according to the invention are administered in combination with a lipoprotein(a) antagonist, by way of example and with preference gemcabene calcium (CI-1027) or nicotinic acid.

The present invention further provides medicaments which comprise at least one compound according to the invention, typically together with one or more inert, non-toxic, pharmaceutically suitable excipients, and for the use thereof for the aforementioned purposes.

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in administration forms suitable for these administration routes.

Suitable administration forms for oral administration are those which work according to the prior art and release the compounds according to the invention rapidly and/or in a modified manner and which contain the compounds according to the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral or parenteral administration, especially oral administration.

The compounds according to the invention can be converted to the administration forms mentioned. This can be accomplished in a manner known per se by mixing with inert, non-toxic, pharmaceutically suitable auxiliaries. These auxiliaries include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration, the dose is about 0.001 to 2 mg/kg, preferably about 0.001 to 1 mg/kg, of body weight.

It may nevertheless be necessary in some cases to deviate from the stated amounts, specifically as a function of body weight, route of administration, individual response to the active compound, nature of the preparation and time or interval over which administration takes place. Thus in some cases it may be sufficient to manage with less than the abovementioned minimum amount, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight. Solvent ratios, dilution ratios and concentration data for the liquid/liquid solutions are based in each case on volume.

### A. EXAMPLES

Abbreviations and Acronyms:

| | |
|---|---|
| abs. | absolute (=dried) |
| aq. | aqueous solution |
| br | broad signal (NMR coupling pattern) |
| CAS No. | Chemical Abstracts Service number |
| δ | shift in the NMR spectrum (stated in ppm) |
| d | doublet (NMR coupling pattern) |
| TLC | thin-layer chromatography |
| DCI | direct chemical ionization (in MS) |
| DMAP | 4-N,N-dimethylaminopyridine |
| DMF | dimethylformamide |
| DMSO | dimethyl sulphoxide |
| ent | enantiomerically pure |
| eq. | equivalent(s) |
| ESI | electrospray ionization (in MS) |
| Et | ethyl |
| h | hour(s) |
| HATU | N-[(dimethylamino)(3H-[1,2,3]triazolo[4,5-b]-pyridin-3- |
| | yloxy)methylene]-N-methylmethanaminium |
| | hexafluorophosphate |
| HPLC | high-pressure, high-performance liquid chromatography |
| HRMS | high-resolution mass spectrometry |
| conc. | concentrated |
| LC-MS | liquid chromatography-coupled mass spectrometry |
| LiHMDS | lithium hexamethyldisilazide |
| m | multiplet (NMR coupling pattern) |
| Me | methyl |
| min | minute(s) |
| MS | mass spectrometry |
| NMR | nuclear magnetic resonance spectrometry |
| Ph | phenyl |
| q | quartet (NMR coupling pattern) |
| quint. | quintet (NMR coupling pattern) |
| rac | racemic |
| R_{F} | retention factor (in thin-layer chromatography) |
| RT | room temperature |
| Rₜ | retention time (in HPLC) |
| s | singlet (NMR coupling pattern) |
| t | triplet (NMR coupling pattern) |
| THF | tetrahydrofuran |
| TBTU | (benzotriazol-1-yloxy)bisdimethylaminomethylium |
| | fluoroborate |
| UV | ultraviolet spectrometry |
| v/v | ratio by volume (of a solution) |

LC/MS and HPLC Methods:

Method 1 (LC-MS):

Instrument: Micromass Quattro Premier with Waters UPLC Acquity; column: Thermo Hypersil GOLD 1.9 μ 50×1 mm; mobile phase A: 1 l of water+0.5 ml of 50% formic acid, mobile phase B: 1 l of acetonitrile+0.5 ml of 50% strength formic acid; gradient: 0.0 min 90% A→0.1 min 90% A→1.5 min 10% A→2.2 min 10% A; oven: 50° C.; flow rate: 0.33 ml/min; UV detection: 210 nm

Method 2 (LC-MS):

Instrument: Waters ACQUITY SQD UPLC system; column: Waters Acquity UPLC HSS T3 1.8 μ 50×1 mm; mobile phase A: 1 l of water+0.25 ml of 99% formic acid, mobile phase B: 1 l of acetonitrile+0.25 ml of 99% strength formic acid; gradient: 0.0 min 90% A→1.2 min 5% A→2.0 min 5% A oven: 50° C.; flow rate: 0.40 ml/min; UV detection: 210-400 nm.

Method 3 (LC-MS):

Instrument: Micromass Quattro Premier with Waters UPLC Acquity; column: Thermo Hypersil GOLD 1.9 μ 50×1 mm; mobile phase A: 1 l of water+0.5 ml of 50% formic acid, mobile phase B: 1 l of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

Method 4 (LC-MS):

MS instrument: Waters (Micromass) QM; HPLC instrument: Agilent 1100 series; column: Agilent ZORBAX Extend-C18 3.0×50 mm 3.5 micron; mobile phase A: 1 l of water+0.01 mol of ammonium carbonate, mobile phase B: 1 l of acetonitrile; gradient: 0.0 min 98% A→0.2 min 98% A→3.0 min 5% A→4.5 min 5% A; oven: 40° C.; flow rate: 1.75 ml/min; UV detection: 210 nm

Method 5 (LC-MS):

Instrument: Waters ACQUITY SQD UPLC system; column: Waters Acquity UPLC HSS T3 1.8 μ 30×2 mm; mobile phase A: 1 l of water+0.25 ml of 99% formic acid, mobile phase B: 1 l of acetonitrile+0.25 ml of 99% strength formic acid; gradient: 0.0 min 90% A→1.2 min 5% A→2.0 min 5% A oven: 50° C.; flow rate: 0.60 ml/min; UV detection: 208-400 nm.

Method 6 (GC-MS):

Instrument: Thermo Scientific DSQII, Thermo Scientific Trace GC Ultra; column: Restek RTX-35MS, 15 m×200 μm×0.33 μm; constant flow rate with helium: 1.20 ml/min; oven: 60° C.; inlet: 220° C.; gradient: 60° C., 30° C./min→300° C. (maintain for 3.33 min).

Unless stated otherwise, the percentages in the tests and examples which follow are percentages by weight; parts are parts by weight. Solvent ratios, dilution ratios and concentration data for the liquid/liquid solutions are based in each case on volume.

The multiplicities of proton signals in ¹H NMR spectra reported in the paragraphs which follow represent the signal form observed in each case and do not take account of any higher-order signal phenomena. In all ¹H NMR spectra data, the chemical shifts δ are stated in ppm.

Additionally, the starting materials, intermediates and working examples may be present as hydrates. There was no quantitative determination of the water content. In certain cases, the hydrates may affect the ¹H NMR spectrum and possibly shift and/or significantly broaden the water signal in the ¹H NMR.

In ¹H NMR spectra, the methyl group of the chemical system “2-methylimidazo[1,2-a]pyridine” appears as a singlet (frequently in DMSO-d₆ and in the range of 2.40-2.60 ppm) and is either clearly distinguishable as such, is superposed by the solvent signals or is completely under the signals of the solvents. In the ¹H NMR spectra, this signal can be assumed to be present.

When compounds of the invention are purified by preparative HPLC by the above-described methods in which the eluents contain additives, for example trifluoroacetic acid, formic acid or ammonia, the compounds of the invention may be obtained in salt form, for example as trifluoroacetate, formate or ammonium salt, if the compounds of the invention contain a sufficiently basic or acidic functionality. Such a salt can be converted to the corresponding free base or acid by various methods known to the person skilled in the art.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

Starting Materials and Intermediates:

### Example 1A

### 5-Chloro-2-nitropyridin-3-ol

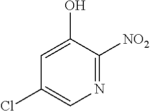 [SMILES: O=[N+]([O-])c1ncc(Cl)cc1O]

With ice cooling, 30 g of 5-chloropyridin-3-ol (232 mmol, 1 equivalent) were dissolved in 228 ml of concentrated sulphuric acid, and 24 ml of concentrated nitric acid were added slowly at 0° C. The reaction was warmed to RT, stirred overnight and then stirred into an ice/water mixture and stirred for another 30 min. The solid was filtered off, washed with cold water and air-dried. This gave 33 g (82% of theory) of the title compound which was used without further purification for the next reaction.

LC-MS (Method 2): Rₜ=0.60 min

MS (ESneg): m/z=172.9/174.9 (M−H)⁻

¹H-NMR (400 MHz, DMSO-d₆): δ=7.71 (d, 1H); 8.10 (d, 1H); 12.14 (br. 1H).

### Example 2A

### 5-Chloro-3-[(2,6-difluorobenzyl)oxy]-2-nitropyridine

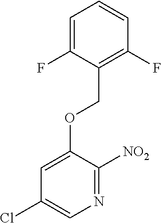 [SMILES: O=[N+]([O-])c1ncc(Cl)cc1OCc1c(F)cccc1F]

33 g of 5-chloro-2-nitropyridin-3-ol (Example 1A; 189 mmol, 1 equivalent) and 61.6 g of caesium carbonate (189 mmol, 1 equivalent) were initially charged in 528 ml of DMF, 40.4 g of 2,6-difluorobenzyl bromide (189 mmol, 1 equivalent) were added and the mixture was stirred at RT overnight. The reaction mixture was stirred into water/1N aqueous hydrochloric acid. The solid was filtered off, washed with water and air-dried. This gave 54.9 g (97% of theory) of the title compound.

¹H-NMR (400 MHz, DMSO-d₆): δ=5.46 (s, 2H); 7.22 (t, 2H); 7.58 (q, 1H); 8.28 (d, 1H); 8.47 (d, 1H).

### Example 3A

### 5-Chloro-3-[(2,6-difluorobenzyl)oxy]pyridine-2-amine

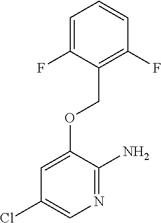 [SMILES: Nc1ncc(Cl)cc1OCc1c(F)cccc1F]

59.7 g of 5-chloro-3-[(2,6-difluorobenzyl)oxy]-2-nitropyridine (199 mmol, 1 equivalent) were initially charged in 600 ml of ethanol, 34.4 g of iron powder (616 mmol, 3.1 equivalents) were added and the mixture was heated to reflux. 152 ml of concentrated hydrochloric acid were slowly added dropwise, and the mixture was boiled at reflux for a further 30 min. The reaction mixture was cooled and stirred into an ice/water mixture. The resulting mixture was adjusted to pH 5 using sodium acetate. The solid was filtered off, washed with water and air-dried and then dried under reduced pressure at 50° C. This gave 52.7 g (98% of theory) of the title compound.

LC-MS (Method 2): Rₜ=0.93 min

MS (ESpos): m/z=271.1/273.1 (M+H)⁺

¹H-NMR (400 MHz, DMSO-d₆): δ=5.14 (s, 2H); 5.82 (br. s, 2H); 7.20 (t, 2H); 7.35 (d, 1H); 7.55 (q, 1H); 7.56 (d, 1H).

### Example 4A

### Ethyl 6-chloro-8-[(2,6-difluorobenzyl)oxy]-2-methylimidazo[1,2-a]pyridine-3-carboxylate

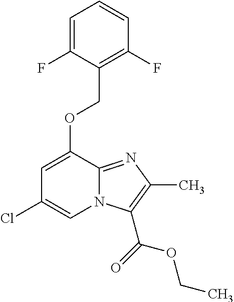 [SMILES: CCOC(=O)c1c(C)nc2c(OCc3c(F)cccc3F)cc(Cl)cn12]

40 g of 5-chloro-3-[(2,6-difluorobenzyl)oxy]pyridine-2-amine (Example 3A; 147.8 mmol, 1 equivalent) were initially charged in 800 ml of ethanol, 30 g of powdered molecular sieve 3 Å and 128 g of ethyl 2-chloroacetoacetate (739 mmol, 5 equivalents) were added and the mixture was heated at reflux overnight. The reaction mixture was concentrated, and the residue was taken up in ethyl acetate and filtered. The ethyl acetate phase was washed with water, dried, filtered and concentrated. This gave 44 g (78% of theory) of the title compound.

LC-MS (Method 2): Rₜ=1.27 min

MS (ESpos): m/z=381.2/383.2 (M+H)⁺

¹H-NMR (400 MHz, DMSO-d₆): δ=1.36 (t, 3H); 2.54 (s, 3H; hidden by DMSO signal); 4.37 (q, 2H); 5.36 (s, 2H); 7.26 (t, 2H); 7.38 (d, 1H); 7.62 (q, 1H); 8.92 (d, 1H).

### Example 5A

### 6-Chloro-8-[(2,6-difluorobenzyl)oxy]-2-methylimidazo[1,2-a]pyridine-3-carboxylic acid

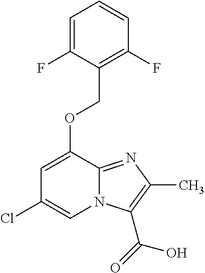 [SMILES: Cc1nc2c(OCc3c(F)cccc3F)cc(Cl)cn2c1C(=O)O]

44 g of ethyl 6-chloro-8-[(2,6-difluorobenzyl)oxy]-2-methylimidazo[1,2-a]pyridine-3-carboxylate (Example 4A; 115 mmol, 1 equivalent) were dissolved in 550 ml of THF and 700 ml of methanol, 13.8 g of lithium hydroxide (dissolved in 150 ml of water; 577 mmol, 5 equivalents) were added and the mixture was stirred at RT overnight. 1 N aqueous hydrochloric acid was added and the mixture was concentrated under reduced pressure. The solid obtained was filtered off and washed with water. This gave 34 g of the title compound (84% of theory).

LC-MS (Method 1): Rₜ=1.03 min

MS (ESpos): m/z=353.0/355.0 (M+H)⁺

¹H-NMR (400 MHz, DMSO-d₆): δ=2.54 (s, 3H; overlapped by DMSO signal); 5.36 (s, 2H); 7.26 (t, 2H); 7.34 (d, 1H); 7.61 (q, 1H); 8.99 (d, 1H); 13.36 (br. s, 1H).

### Example 6A

### 3-[(2,6-Difluorobenzyl)oxy]pyridine-2-amine

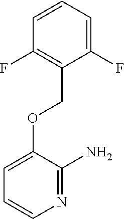 [SMILES: Nc1ncccc1OCc1c(F)cccc1F]

At RT, 51 g of sodium methoxide (953 mmol, 1.05 equivalents) were initially charged in 1000 ml of methanol, 100 g of 2-amino-3-hydroxypyridine (908 mmol, 1 equivalent) were added and the mixture was stirred at RT for another 15 min. The reaction mixture was concentrated under reduced pressure, the residue was taken up in 2500 ml of DMSO and 197 g of 2,6-difluorobenzyl bromide (953 mmol, 1.05 equivalents) were added. After 4 h at RT, the reaction mixture was poured onto 20 l of water, the mixture was stirred for a further 15 min and the solid was filtered off. The solid was washed with 1 l of water and 100 ml of isopropanol and 500 ml of petroleum ether and dried under high vacuum. This gave 171 g of the title compound (78% of theory).

¹H-NMR (400 MHz, DMSO-d₆): δ=5.10 (s, 2H); 5.52 (br. s, 2H), 6.52 (dd, 1H); 7.16-7.21 (m, 3H); 7.49-7.56 (m, 2H).

### Example 7A

### Ethyl 8-[(2,6-difluorobenzyl)oxy]-2-methylimidazo[1,2-a]pyridine-3-carboxylate

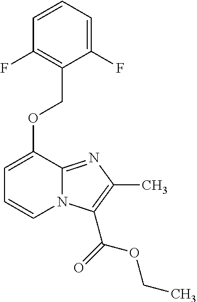 [SMILES: CCOC(=O)c1c(C)nc2c(OCc3c(F)cccc3F)cccn12]

170 g of 3-[(2,6-difluorobenzyl)oxy]pyridine-2-amine (Example 6A; 719 mmol, 1 equivalent) were initially charged in 3800 ml of ethanol, and 151 g of powdered molecular sieve 3 Å and 623 g of ethyl 2-chloroacetoacetate (3.6 mol, 5 equivalents) were added. The reaction mixture was heated at reflux for 24 h and then filtered off through silica gel and concentrated under reduced pressure. The mixture was kept at RT for 48 h and the solid formed was filtered off. The solid was then stirred three times with a little isopropanol and then filtered off, and washed with diethyl ether. This gave 60.8 g (23% of theory) of the title compound. The combined filtrates of the filtration steps were concentrated and the residue was chromatographed on silica gel using the mobile phase cyclohexane/diethyl ether. This gave a further 46.5 g (18% of theory; total yield: 41% of theory) of the title compound.

LC-MS (Method 2): Rₜ=1.01 min

MS (ESpos): m/z=347 (M+H)⁺

¹H-NMR (400 MHz, DMSO-d₆): δ=1.36 (t, 3H); 2.54 (s, 3H; hidden by DMSO signal); 4.36 (q, 2H); 5.33 (s, 2H); 7.11 (t, 1H); 7.18-7.27 (m, 3H); 7.59 (quint, 1H); 8.88 (d, 1H).

### Example 8A

### 8-[(2,6-Difluorobenzyl)oxy]-2-methylimidazo[1,2-a]pyridine-3-carboxylic acid

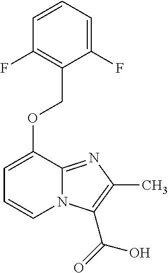 [SMILES: Cc1nc2c(OCc3c(F)cccc3F)cccn2c1C(=O)O]

107 g of ethyl 8-[(2,6-difluorobenzyl)oxy]-2-methylimidazo[1,2-a]pyridine-3-carboxylate (Example 7A; 300 mmol, 1 equivalent) were dissolved in 2.8 l of THF/methanol (1:1), 1.5 l of 1 N aqueous lithium hydroxide solution (1.5 mol, 5 equivalents) were added and the mixture was stirred at RT for 16 h. The organic solvents were removed under reduced pressure and the resulting aqueous solution was, in an ice bath, adjusted to pH 3-4 using 1 N aqueous hydrochloric acid. The resulting solid was filtered off, washed with water and isopropanol and dried under reduced pressure. This gave 92 g (95% of theory) of the title compound.

LC-MS (Method 2): Rₜ=0.62 min

MS (ESpos): m/z=319.1 (M+H)⁺

¹H-NMR (400 MHz, DMSO-d₆): δ=2.55 (s, 3H; overlapped by DMSO signal); 5.32 (s, 2H); 7.01 (t, 1H); 7.09 (d, 1H); 7.23 (t, 2H); 7.59 (quint, 1H); 9.01 (d, 1H).

### Example 9A

### rac-2-Amino-5,5,5-trifluoro-2-methylpentanonitrile

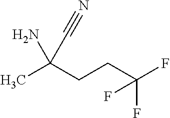

8.0 g (57.1 mmol) of 5,5,5-trifluoropentan-2-one [CAS Registry Number: 1341078-97-4; commercially available, or the methyl ketone can be prepared by literature methods which are known to those skilled in the art, for example via a) two stages from 4,4,4-trifluorobutanal according to Y. Bai et al. Angewandte Chemie 2012, 51, 4112-4116; K. Hiroi et al. Synlett 2001, 263-265; K. Mikami et al. 1982 Chemistry Letters, 1349-1352; b) or from 4,4,4-trifluorobutanoic acid according to A. A. Wube et al. Bioorganic and Medicinal Chemistry 2011, 19, 567-579; G. M. Rubottom et al. Journal of Organic Chemistry 1983, 48, 1550-1552; T. Chen et al. Journal of Organic Chemistry 1996, 61, 4716-4719. The product can be isolated by distillation or chromatography.] were initially charged in 47.8 ml of 2 N ammonia in methanol, 3.69 g (75.4 mmol) of sodium cyanide and 4.03 g (75.4 mmol) of ammonium chloride were added at room temperature and the mixture was stirred under reflux for 4 hours. The reaction mixture was cooled, diethyl ether was added and the solids present were filtered off. The solvent was distilled out of the filtrate under standard pressure. 8.7 g of the title compound (92% of theory) were obtained as residue, which was used in the subsequent stage without further purification.

GC-MS (Method 6): Rₜ=1.90 min

MS (ESpos): m/z=151 (M-CH₃)⁺

### Example 10A

### rac-Benzyl (2-cyano-5,5,5-trifluoropentan-2-yl)carbamate

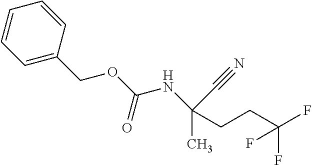 [SMILES: CC(C#N)(CCC(F)(F)F)NC(=O)OCc1ccccc1]

8.7 g (52.36 mmol) of rac-2-amino-5,5,5-trifluoro-2-methylpentanonitrile from Example 9A were initially charged in 128 ml of tetrahydrofuran/water=9/1, and 22.43 g (162.3 mmol) of potassium carbonate were added. At 0° C., 8.93 g (52.36 mmol) of benzyl chloroformate were slowly added dropwise. Then the mixture was allowed to warm up gradually to room temperature and stirred at room temperature overnight. The supernatant solvent was decanted off, the residue was twice stirred with 100 ml each time of tetrahydrofuran, and then the supernatant solvent was decanted off each time. The combined organic phases were concentrated and the crude product was purified by silica gel chromatography (mobile phase: cyclohexane/ethyl acetate gradient 9/1 to 4/1). 11.14 g of the title compound (68% of theory) were obtained.

LC-MS (Method 2): Rₜ=1.01 min

MS (ESpos): m/z=301 (M+H)⁺

¹H-NMR (400 MHz, DMSO-d₆): δ [ppm]=1.58 (s, 3H), 2.08-2.21 (m, 2H), 2.24-2.52 (m, 2H), 5.09 (s, 2H), 7.29-7.41 (m, 5H), 8.17 (br. s, 1H).

### Example 11A

### ent-Benzyl (2-cyano-5,5,5-trifluoropentan-2-yl)carbamate (enantiomer A)

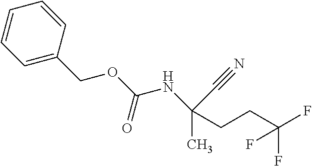 [SMILES: CC(C#N)(CCC(F)(F)F)NC(=O)OCc1ccccc1]

11.14 g of rac-benzyl (2-cyano-5,5,5-trifluoropentan-2-yl)carbamate from Example 10A were separated into the enantiomers by preparative separation on a chiral phase [column: Daicel Chiralpak AZ-H, 5 μm, SFC, 250×50 mm, mobile phase: 94% carbon dioxide, 6% methanol, flow rate: 200 ml/min, temperature: 38° C., pressure: 135 bar; detection: 210 nm].

enantiomer A: 4.12 g (about 79% ee)

Rₜ=1.60 min [SFC, Daicel Chiralpak AZ-H, 250×4.6 mm, 5 μm, mobile phase: 90% carbon dioxide, 10% methanol, flow rate: 3 ml/min, temperature: 30° C., detection: 220 nm].

LC-MS (Method 2): Rₜ=1.01 min

MS (ESpos): m/z=301 (M+H)⁺

### Example 12A

### ent-Benzyl (2-cyano-5,5,5-trifluoropentan-2-yl)carbamate (enantiomer B)

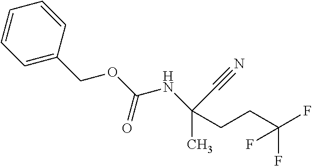 [SMILES: CC(C#N)(CCC(F)(F)F)NC(=O)OCc1ccccc1]

11.14 g of rac-benzyl (2-cyano-5,5,5-trifluoropentan-2-yl)carbamate from Example 10A were separated into the enantiomers by preparative separation on a chiral phase [column: Daicel Chiralpak AZ-H, 5 μm, SFC, 250×50 mm, mobile phase: 94% carbon dioxide, 6% methanol, flow rate: 200 ml/min, temperature: 38° C., pressure: 135 bar; detection: 210 nm].

enantiomer B: 4.54 g (about 70% ee, about 89% purity)

Rₜ=1.91 min [SFC, Daicel Chiralpak AZ-H, 250×4.6 mm, 5 μm, mobile phase: 90% carbon dioxide, 10% methanol, flow rate: 3 ml/min, temperature: 30° C., detection: 220 nm].

LC-MS (Method 2): Rₜ=1.01 min

MS (ESpos): m/z=301 (M+H)⁺

### Example 13A

### ent-Benzyl (1-amino-5,5,5-trifluoro-2-methylpentan-2-yl)carbamate (enantiomer A)

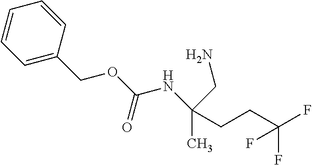 [SMILES: CC(CN)(CCC(F)(F)F)NC(=O)OCc1ccccc1]

4.12 g (13.17 mmol) of ent-benzyl (2-cyano-5,5,5-trifluoropentan-2-yl)carbamate (enantiomer A) from Example 11A were dissolved in 39 ml of 7 N ammonia solution in methanol, and 4 g of Raney nickel (50% aqueous slurry) were added under argon. The reaction mixture was hydrogenated in an autoclave at 20-30 bar overnight. Another 1 g of Raney nickel (50% aqueous slurry) was added and the reaction mixture was hydrogenated in an autoclave at 20-30 bar for 5 h. The reaction mixture was filtered through kieselguhr, rinsed with methanol and concentrated. 3.35 g (56% of theory; purity about 67%) of the target compound were obtained, which were used in the subsequent stage without further purification.

LC-MS (Method 5): Rₜ=1.68 min

MS (ESpos): m/z=305 (M+H)⁺

¹H-NMR (400 MHz, DMSO-d₆): δ [ppm]=1.13 (s, 3H), 1.40 (br. s, 2H), 1.70-1.80 (m, 1H), 1.83-1.95 (m, 1H), 2.08-2.2 (m, 2H), 4.98 (s, 2H), 6.85 (br. s, 1H), 7.28-7.41 (m, 5H).

### Example 14A

### ent-Benzyl (1-amino-5,5,5-trifluoro-2-methylpentan-2-yl)carbamate (enantiomer B)

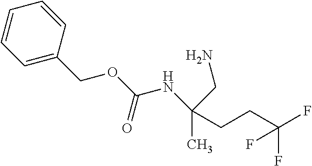 [SMILES: CC(CN)(CCC(F)(F)F)NC(=O)OCc1ccccc1]

4.54 g (13.45 mmol; purity about 89%) of ent-benzyl (2-cyano-5,5,5-trifluoropentan-2-yl)carbamate (enantiomer B) from Example 12A were dissolved in 39 ml of 7 N ammonia solution in methanol, and 5 g of Raney nickel (50% aqueous slurry) were added under argon. The reaction mixture was hydrogenated in an autoclave at 20-30 bar for 3 h. The reaction mixture was filtered through kieselguhr, rinsed with methanol and concentrated. 4.20 g (97% of theory; purity about 95%) of the target compound were obtained, which were used in the subsequent stage without further purification.

LC-MS (Method 4): Rₜ=2.19 min

MS (ESpos): m/z=305 (M+H)⁺

¹H-NMR (400 MHz, DMSO-d₆): δ [ppm]=1.13 (s, 3H), 1.40 (br. s, 2H), 1.69-1.80 (m, 1H), 1.83-1.96 (m, 1H), 2.07-2.22 (m, 2H), 4.98 (s, 2H), 6.85 (br. s, 1H), 7.27-7.40 (m, 5H).

### Example 15A

### rac-2-[(Diphenylmethylene)amino]-4,4-difluorobutanonitrile

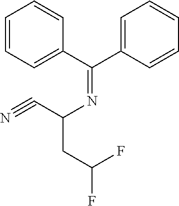 [SMILES: N#CC(CC(F)F)N=C(c1ccccc1)c1ccccc1]

18 g (81.72 mmol) of [(diphenylmethylene)amino]acetonitrile were initially charged in 500 ml of abs. THF, and 39.22 ml (98.06 mmol) of n-butyllithium (2.5 N in hexane) were added at −78° C. under argon, and the mixture was stirred at −78° C. for 15 min. Subsequently, the reaction solution was warmed up to 0° C. 17.25 g (89.89 mmol) of 1,1-difluoro-2-iodoethane were added dropwise, and the mixture was stirred at 0° C. for a further 15 min. At 0° C., first water and then ethyl acetate were added to the reaction solution, and the mixture was washed three times with semisaturated aqueous sodium chloride solution. The combined aqueous phases were re-extracted twice with ethyl acetate. The combined organic phases were dried over sodium sulphate, filtered and concentrated. The residue was purified by silica gel chromatography (mobile phase: dichloromethane/cyclohexane=1/1). This gave 13.57 g of the target compound (49% of theory, purity 84%).

LC-MS (Method 3): Rₜ=2.48 min

MS (ESpos): m/z=285 (M+H)⁺

¹H-NMR (400 MHz, DMSO-d₆): δ=2.53-2.61 (m, 2H; partially overlapped with solvent peak), 4.50 (t, 1H), 6.08-6.41 (m, 1H), 7.23-7.33 (m, 2H), 7.38-7.47 (m, 2H), 7.49-7.67 (m, 6H).

### Example 16A

### rac-2-[(Diphenylmethylene)amino]-5-fluoropentanonitrile

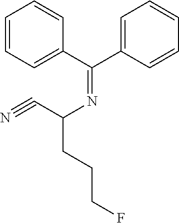 [SMILES: N#CC(CCCF)N=C(c1ccccc1)c1ccccc1]

To an initial charge of 18 g (81.72 mmol) of [(diphenylmethylene)amino]acetonitrile in 500 ml of abs. THF were added 39.22 ml (98.06 mmol) of n-butyllithium (2.5 N in hexane) at −78° C. under argon, and the mixture was stirred at −78° C. for a further 15 min. Subsequently, the reaction solution was warmed up to 0° C. and 16.9 g (89.89 mmol) of 1-fluoro-3-iodopropane were added dropwise to the reaction solution, which was stirred at 0° C. for a further 15 min. At 0° C., first water and then ethyl acetate were added to the reaction solution, and the mixture was washed with saturated aqueous sodium chloride solution. The aqueous phase was extracted twice with ethyl acetate. The combined organic phases were dried over sodium sulphate, filtered and concentrated. The residue was purified by silica gel chromatography (mobile phase: toluene 100%, re-purification with dichloromethane/cyclohexane=1/1 to 2/1). This gave 16.73 g of the target compound (73% of theory).

LC-MS (Method 3): Rₜ=2.50 min

MS (ESpos): m/z=281 (M+H)⁺

¹H-NMR (400 MHz, DMSO-d₆): δ=1.66-1.85 (m, 2H), 1.87-2.00 (m, 2H), 4.26-4.41 (m, 2H), 4.43-4.55 (m, 1H), 7.20-7.33 (m, 2H), 7.38-7.48 (m, 2H), 7.48-7.63 (m, 6H).

### Example 17A

### rac-2-[(Diphenylmethylene)amino]-4,4-difluoro-2-methylbutanonitrile

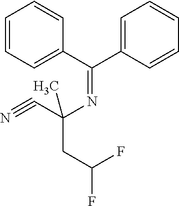 [SMILES: CC(C#N)(CC(F)F)N=C(c1ccccc1)c1ccccc1]

To an initial charge of 13.07 g (38.62 mmol) of rac-2-[(diphenylmethylene)amino]-4,4-difluorobutanonitrile from Example 15A in 255 ml of abs. THF were added 15.6 ml (39.0 mmol) of n-butyllithium (2.5 N in hexane) at −78° C. under argon, and the mixture was stirred at −78° C. for a further 10 min. Subsequently, 22.6 g (154.46 mmol) of iodomethane were added to the reaction solution at −78° C. The reaction mixture was gradually brought to 0° C. over 3.5 h. At 0° C., first water and then ethyl acetate were added and the mixture was washed twice with saturated aqueous sodium chloride solution. The organic phase was dried over sodium sulphate, filtered and concentrated. The residue was purified by silica gel chromatography (mobile phase: cyclohexane/ethyl acetate=15/1). This gave 11.4 g of the target compound (91% of theory, purity 92%).

LC-MS (Method 3): Rₜ=2.52 min

MS (ESpos): m/z=299 (M+H)⁺

¹H-NMR (400 MHz, DMSO-d₆): δ=1.67 (s, 3H), 2.55-2.77 (m, 2H), 6.14-6.48 (m, 1H), 7.28-7.34 (m, 2H), 7.36-7.44 (m, 2H), 7.44-7.54 (m, 6H).

### Example 18A

### rac-2-[(Diphenylmethylene)amino]-5-fluoro-2-methylpentanonitrile

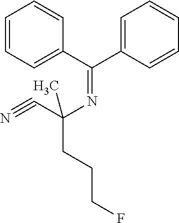 [SMILES: CC(C#N)(CCCF)N=C(c1ccccc1)c1ccccc1]

To an initial charge of 16.73 g (59.68 mmol) of rac-2-[(diphenylmethylene)amino]-5-fluoropentanonitrile from Example 16A in 394 ml of abs. THF were added 24.11 ml (60.27 mmol) of n-butyllithium (2.5 N in hexane) at −78° C. under argon, and the mixture was stirred at −78° C. for a further 10 min. Subsequently, 34.93 g (238.70 mmol) of iodomethane were added to the reaction solution at −78° C. The reaction mixture was gradually brought to 0° C. over 4.5 h. At 0° C., first water and then ethyl acetate were added and the mixture was washed twice with saturated aqueous sodium chloride solution. The organic phase was dried over sodium sulphate, filtered and concentrated. The residue was purified by silica gel chromatography (mobile phase: cyclohexane/ethyl acetate=15/1). This gave 18.94 g of the target compound (95% of theory, purity 88%).

LC-MS (Method 3): Rₜ=2.55 min

MS (ESpos): m/z=295 (M+H)⁺

¹H-NMR (400 MHz, DMSO-d₆): δ=1.62 (s, 3H), 1.73-1.90 (m, 2H), 1.94-2.03 (m, 1H), 2.04-2.18 (m, 1H), 4.47 (t, 1H), 4.58 (t, 1H), 7.23-7.33 (m, 2H), 7.35-7.43 (m, 2H), 7.44-7.56 (m, 6H).

### Example 19A

### rac-2-Amino-4,4-difluoro-2-methylbutanonitrile hydrochloride

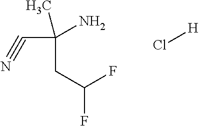

10.84 g (33.43 mmol; 92% purity) of rac-2-[(diphenylmethylene)amino]-4,4-difluoro-2-methylbutanonitrile from Example 17A were dissolved in 156 ml of tetrahydrofuran and 6 ml of water, 73.5 ml (36.77 mmol) of hydrogen chloride solution (0.5 N in diethyl ether) were added and the mixture was stirred at room temperature overnight. 16.71 ml (33.43 mmol) of hydrogen chloride solution (2 N in diethyl ether) were then added to the reaction solution, and the mixture was concentrated. The isolated crude product was reacted further directly without further purification.

LC-MS (Method 3): Rₜ=0.32 min

MS (ESpos): m/z=135 (M-HCl+H)⁺

### Example 20A

### rac-Benzyl (2-cyano-4,4-difluorobutan-2-yl)carbamate

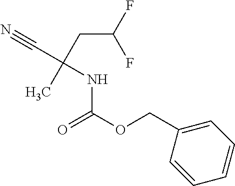 [SMILES: CC(C#N)(CC(F)F)NC(=O)OCc1ccccc1]

The crude product rac-2-amino-4,4-difluoro-2-methylbutanonitrile hydrochloride from Example 19A was initially charged in 109 ml of tetrahydrofuran/water (1:1), and 18.94 g (137.06 mmol) of potassium carbonate and 6.27 g (36.77 mmol) of benzyl chloroformate were added. The reaction mixture was stirred at room temperature overnight. Another 1.14 g (6.69 mmol) of benzyl chloroformate were added to the reaction and the mixture was stirred at room temperature for a further 2 h. The phases were then separated and the aqueous phase was extracted twice with ethyl acetate. The combined organic phases were washed once with saturated aqueous sodium chloride solution, and then dried over sodium sulphate, filtered and concentrated. The residue was purified by silica gel chromatography (mobile phase: cyclohexane/ethyl acetate gradient 20/1 to 5/1). 7.68 g of the target compound were obtained (61% of theory over two stages, 71% purity).

LC-MS (Method 3): Rₜ=2.04 min

MS (ESpos): m/z=269 (M+H)⁺

¹H-NMR (400 MHz, DMSO-d₆): δ [ppm]=1.65 (s, 3H), 2.51-2.65 (m, 2H), 5.10 (s, 2H), 6.08-6.41 (m, 1H), 7.27-7.44 (m, 5H), 8.24 (br. s, 1H).

### Example 21A

### rac-2-Amino-5-fluoro-2-methylpentanonitrile hydrochloride

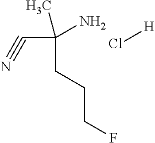

18.94 g (56.62 mmol; 88% purity) of rac-2-[(diphenylmethylene)amino]-5-fluoro-2-methylpentanonitrile from Example 18A were dissolved in 264.6 ml of tetrahydrofuran and 10.2 ml of water, 124.6 ml (62.28 mmol) of hydrogen chloride solution (0.5 N in diethyl ether) were added and the mixture was stirred at room temperature overnight. 28.3 ml (56.62 mmol) of hydrogen chloride solution (2 N in diethyl ether) were then added to the reaction solution, and the mixture was concentrated. The isolated crude product was reacted further directly without further purification.

LC-MS (Method 3): Rₜ=0.25 min

MS (ESpos): m/z=131 (M-HCl+H)⁺

### Example 22A

### rac-Benzyl (2-cyano-5-fluoropentan-2-yl)carbamate

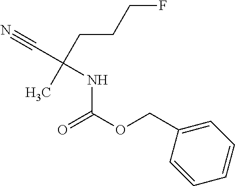 [SMILES: CC(C#N)(CCCF)NC(=O)OCc1ccccc1]

The crude product rac-2-amino-5-fluoro-2-methylpentanonitrile hydrochloride from Example 21A was initially charged in 185 ml of tetrahydrofuran/water (1/1), and 32.09 g (232.18 mmol) of potassium carbonate and 10.63 g (62.29 mmol) of benzyl chloroformate were added. The reaction mixture was stirred at room temperature overnight. Another 1.93 g (11.33 mmol) of benzyl chloroformate were added to the reaction and the mixture was stirred at room temperature for a further 2 h. The phases were then separated and the aqueous phase was extracted twice with ethyl acetate. The combined organic phases were washed once with saturated aqueous sodium chloride solution, and then dried over sodium sulphate, filtered and concentrated. The residue was purified by silica gel chromatography (mobile phase: cyclohexane/ethyl acetate gradient 20/1 to 5/1). This gave 11.77 g of the target compound (72% of theory over two steps, purity 92%).

LC-MS (Method 3): Rₜ=2.03 min

MS (ESpos): m/z=265 (M+H)⁺

¹H-NMR (400 MHz, DMSO-d₆): δ [ppm]=1.55 (s, 3H), 1.66-1.85 (m, 2H), 1.86-2.04 (m, 2H), 4.40 (t, 1H), 4.52 (t, 1H), 5.08 (s, 2H), 7.28-7.44 (m, 5H), 8.05 (br. s, 1H).

### Example 23A

### ent-Benzyl (2-cyano-4,4-difluorobutan-2-yl)carbamate (enantiomer A)

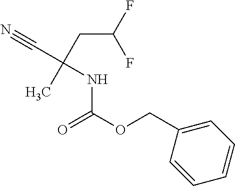 [SMILES: CC(C#N)(CC(F)F)NC(=O)OCc1ccccc1]

7.68 g (20.33 mmol, purity 71%) of rac-benzyl (2-cyano-4,4-difluorobutan-2-yl)carbamate from Example 20A were separated into the enantiomers by preparative separation on a chiral phase [column: Daicel Chiralpak AY-H, 5 μm, 250×20 mm, mobile phase: 80% isohexane, 20% isopropanol; flow rate: 25 ml/min; temperature: 22° C., detection: 210 nm].

Enantiomer A: yield: 2.64 g (>99% ee)

Rₜ=6.67 min [Chiralpak AY-H, 5 μm, 250×4.6 mm; mobile phase: 80% isohexane, 20% isopropanol; flow rate: 3 ml/min; detection: 220 nm].

### Example 24A

### ent-Benzyl (2-cyano-4,4-difluorobutan-2-yl)carbamate (enantiomer B)

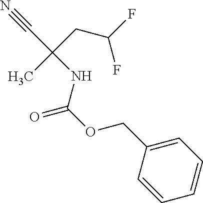 [SMILES: CC(C#N)(CC(F)F)NC(=O)OCc1ccccc1]

7.68 g (20.33 mmol, purity 71%) of rac-benzyl (2-cyano-4,4-difluorobutan-2-yl)carbamate from Example 20A were separated into the enantiomers by preparative separation on a chiral phase [column: Daicel Chiralpak AY-H, 5 μm, 250×20 mm, mobile phase: 80% isohexane, 20% isopropanol; flow rate: 25 ml/min; temperature: 22° C., detection: 210 nm].

Enantiomer B: yield: 2.76 g (93% ee)

Rₜ=7.66 min [Chiralpak AY-H, 5 μm, 250×4.6 mm; mobile phase: 80% isohexane, 20% isopropanol; flow rate: 3 ml/min; detection: 220 nm].

### Example 25A

### ent-Benzyl (2-cyano-5-fluoropentan-2-yl)carbamate (enantiomer A)

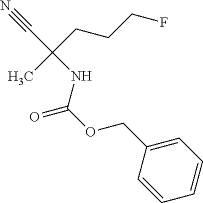 [SMILES: CC(C#N)(CCCF)NC(=O)OCc1ccccc1]

11.77 g (40.97 mmol, 92% purity) of rac-benzyl (2-cyano-5-fluoropentan-2-yl)carbamate from Example 22A were separated into the enantiomers by preparative separation on a chiral phase [column: SFC Daicel Chiralpak AZ-H, 5 μm, 250×30 mm, mobile phase: 90% CO₂, 10% methanol, flow rate: 100 ml/min; temperature: 40° C., detection: 210 nm].

Enantiomer A: yield: 5.7 g (>99% ee)

Rₜ=1.76 min [SFC, Chiralpak AZ-3, 3 μm, 50×4.6 mm; mobile phase: CO₂/methanol gradient (5% to 60% methanol); flow rate: 3 ml/min; detection: 220 nm].

### Example 26A

### ent-Benzyl (2-cyano-5-fluoropentan-2-yl)carbamate (enantiomer B)

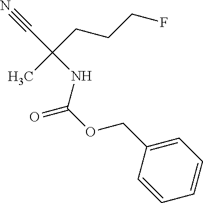 [SMILES: CC(C#N)(CCCF)NC(=O)OCc1ccccc1]

11.77 g (40.97 mmol, purity 92%) of rac-benzyl (2-cyano-5-fluorobutan-2-yl)carbamate from Example 22A were separated into the enantiomers by preparative separation on a chiral phase [column: SFC Daicel Chiralpak AZ-H, 5 μm, 250×30 mm, mobile phase: 90% CO₂, 10% methanol, flow rate: 100 ml/min; temperature: 40° C., detection: 210 nm].

Enantiomer B: yield: 5.0 g (>99% ee)

Rₜ=1.97 min [SFC, Chiralpak AZ-3, 3 μm, 50×4.6 mm; mobile phase: CO₂/methanol gradient (5% to 60% methanol); flow rate: 3 ml/min; detection: 220 nm].

### Example 27A

### ent-Benzyl (1-amino-4,4-difluoro-2-methylbutan-2-yl)carbamate (enantiomer A)

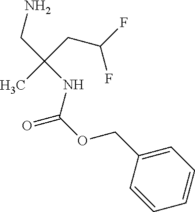 [SMILES: CC(CN)(CC(F)F)NC(=O)OCc1ccccc1]

2.3 g (8.57 mmol) of ent-benzyl (2-cyano-4,4-difluorobutan-2-yl)carbamate (enantiomer A) from Example 23A were dissolved in 75 ml of 7 N ammonia solution in methanol, and 2.66 g of Raney nickel (50% aqueous slurry) were added under argon. The reaction mixture was hydrogenated in an autoclave at 20-30 bar for 1.5 h. The reaction mixture was filtered through Celite, rinsed with methanol and 2 N ammonia in methanol, and concentrated. This gave 2.23 g of the target compound (94% of theory).

LC-MS (Method 3): Rₜ=1.48 min

MS (ESpos): m/z=273 (M+H)⁺

¹H-NMR (400 MHz, DMSO-d₆): δ [ppm]=1.19 (s, 3H), 1.48 (br. s, 2H), 2.08-2.40 (m, 2H), 2.53-2.72 (m, 2H; partially overlapped with solvent peak), 5.00 (s, 2H), 5.90-6.23 (m, 1H), 6.95 (br. s, 1H), 7.25-7.41 (m, 5H).

### Example 28A

### ent-Benzyl (1-amino-4,4-difluoro-2-methylbutan-2-yl)carbamate (enantiomer B)

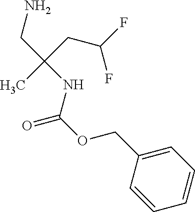 [SMILES: CC(CN)(CC(F)F)NC(=O)OCc1ccccc1]

2.76 g (10.29 mmol) of ent-benzyl (2-cyano-4,4-difluorobutan-2-yl)carbamate (enantiomer B) from Example 24A were dissolved in 90 ml of 7 N ammonia solution in methanol, and 3.19 g of Raney nickel (50% aqueous slurry) were added under argon. The reaction mixture was hydrogenated in an autoclave at 20-30 bar for 1.5 h. The reaction mixture was filtered through Celite, rinsed with methanol and 2 N ammonia in methanol, and concentrated. This gave 2.64 g of the target compound (88% of theory, purity 93%).

LC-MS (Method 3): Rₜ=1.49 min

MS (ESpos): m/z=273 (M+H)⁺

¹H-NMR (400 MHz, DMSO-d₆): δ [ppm]=1.19 (s, 3H), 1.48 (br. s, 2H), 2.08-2.40 (m, 2H), 2.53-2.73 (m, 2H; partially overlapped with solvent peak), 5.00 (s, 2H), 5.90-6.24 (m, 1H), 6.95 (br. s, 1H), 7.25-7.41 (m, 5H).

### Example 29A

### ent-Benzyl (1-amino-5-fluoro-2-methylpentan-2-yl)carbamate (enantiomer A)

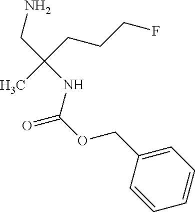 [SMILES: CC(CN)(CCCF)NC(=O)OCc1ccccc1]

5.7 g (21.57 mmol) of ent-benzyl (2-cyano-5-fluoropentan-2-yl)carbamate (enantiomer A) from Example 25A were dissolved in 125 ml of 7 N ammonia solution in methanol, and 6.68 g of Raney nickel (50% aqueous slurry) were added under argon. The reaction mixture was hydrogenated in an autoclave at 20-30 bar for 4.5 h. The reaction mixture was filtered through Celite, rinsed with methanol and 2 N ammonia in methanol, and concentrated. This gave 5.22 g of the target compound (77% of theory, purity 85%).

LC-MS (Method 3): Rₜ=1.51 min

MS (ESpos): m/z=269 (M+H)⁺

### Example 30A

### ent-Benzyl (1-amino-5-fluoro-2-methylpentan-2-yl)carbamate (enantiomer B)

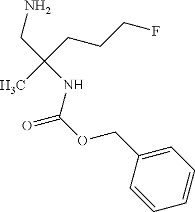 [SMILES: CC(CN)(CCCF)NC(=O)OCc1ccccc1]

5.0 g (18.92 mmol) of ent-benzyl (2-cyano-5-fluoropentan-2-yl)carbamate (enantiomer B) from Example 26A were dissolved in 110 ml of 7 N ammonia solution in methanol, and 5.86 g of Raney nickel (50% aqueous slurry) were added under argon. The reaction mixture was hydrogenated in an autoclave at 20-30 bar for 4.5 h. The reaction mixture was filtered through Celite, rinsed with methanol and 2 N ammonia in methanol, and concentrated. This gave 4.6 g of the target compound (84% of theory, purity 93%).

LC-MS (Method 3): Rₜ=1.47 min

MS (ESpos): m/z=269 (M+H)⁺

### Example 31A

### ent-Benzyl {1-[({6-chloro-8-[(2,6-difluorobenzyl)oxy]-2-methylimidazo[1,2-a]pyridin-3-yl}carbonyl)amino]-5-fluoro-2-methylpentan-2-yl}carbamate trifluoroacetate (enantiomer A)

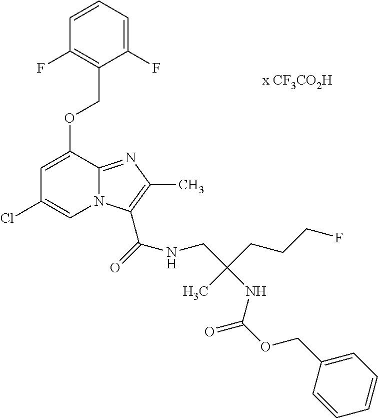 [SMILES: Cc1nc2c(OCc3c(F)cccc3F)cc(Cl)cn2c1C(=O)NCC(C)(CCCF)NC(=O)OCc1ccccc1]

250 mg (0.71 mmol) of 6-chloro-8-[(2,6-difluorobenzyl)oxy]-2-methylimidazo[1,2-a]pyridine-3-carboxylic acid from Example 5A were dissolved in 2.36 ml of DMF, 350 mg (0.92 mmol) of HATU and 0.62 ml (3.54 mmol) of N,N-diisopropylethylamine were added and the mixture was stirred at room temperature for 20 min. 291 mg (0.92 mmol, 85% purity) of ent-benzyl (1-amino-5-fluoro-2-methylpentan-2-yl)carbamate (enantiomer A) from Example 29A were then added. After 30 min, water was added and the resulting solid was filtered off and washed with water. The residue was purified by preparative HPLC (RP 18 column, mobile phase: acetonitrile/water gradient with addition of 0.1% TFA). The product fractions were combined and concentrated. This gave 397 mg of the title compound (71% of theory; purity 91%).

LC-MS (Method 2): Rₜ=1.21 min

MS (ESpos): m/z=603 (M-TFA+H)⁻

¹-NMR (500 MHz, DMSO-d₆): δ [ppm]=1.22 (s, 3H), 1.52-1.75 (m, 3H), 1.80-1.93 (m, 1H), 2.53 (s, 3H; overlapped with solvent peak), 3.49-3.61 (m, 2H), 4.29-4.38 (m, 1H), 4.39-4.51 (m, 1H), 5.00 (s, 2H), 5.37 (s, 2H), 7.07-7.17 (m, 1H), 7.20-7.38 (m, 8H), 7.54-7.64 (m, 1H), 7.90 (t, 1H), 8.76 (d, 1H).

### Example 32A

### ent-Benzyl {1-[({6-chloro-8-[(2,6-difluorobenzyl)oxy]-2-methylimidazo[1,2-a]pyridin-3-yl}carbonyl)amino]-5-fluoro-2-methylpentan-2-yl}carbamate trifluoroacetate (enantiomer B)

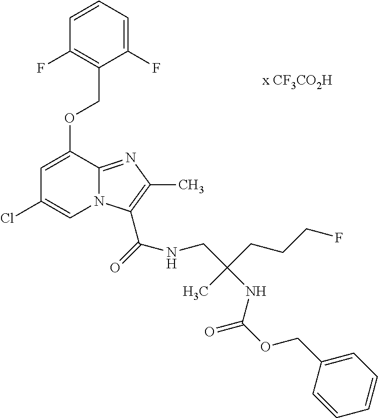 [SMILES: Cc1nc2c(OCc3c(F)cccc3F)cc(Cl)cn2c1C(=O)NCC(C)(CCCF)NC(=O)OCc1ccccc1]

250 mg (0.71 mmol) of 6-chloro-8-[(2,6-difluorobenzyl)oxy]-2-methylimidazo[1,2-a]pyridine-3-carboxylic acid from Example 5A were dissolved in 2.36 ml of DMF, 350 mg (0.92 mmol) of HATU and 0.62 ml (3.54 mmol) of N,N-diisopropylethylamine were added and the mixture was stirred at room temperature for 20 min. Subsequently, 267 mg (0.92 mmol, 93% purity) of ent-benzyl (1-amino-5-fluoro-2-methylpentan-2-yl)carbamate (enantiomer B) from Example 30A were added. After 30 min, water was added and the resulting solid was filtered off and washed with water. The residue was purified by preparative HPLC (RP18 column, mobile phase: acetonitrile/water gradient with addition of 0.1% TFA). The product fractions were combined and concentrated. This gave 328 mg of the title compound (61% of theory; purity 94%).

LC-MS (Method 2): Rₜ=1.21 min

MS (ESpos): m/z=603 (M-TFA+H)⁻

### Example 33A

### ent-Benzyl {1-[({6-chloro-8-[(2,6-difluorobenzyl)oxy]-2-methylimidazo[1,2-a]pyridin-3-yl}carbonyl)amino]-4,4-difluoro-2-methylbutan-2-yl}carbamate trifluoroacetate (enantiomer A)

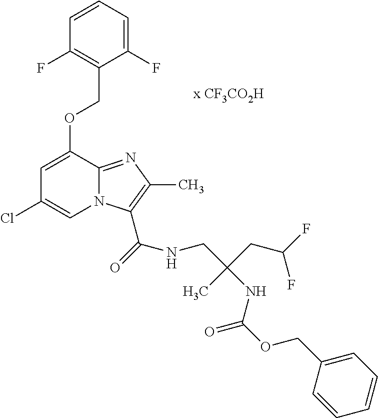 [SMILES: Cc1nc2c(OCc3c(F)cccc3F)cc(Cl)cn2c1C(=O)NCC(C)(CC(F)F)NC(=O)OCc1ccccc1]

250 mg (0.71 mmol) of 6-chloro-8-[(2,6-difluorobenzyl)oxy]-2-methylimidazo[1,2-a]pyridine-3-carboxylic acid from Example 5A were dissolved in 2.36 ml of DMF, 350 mg (0.92 mmol) of HATU and 0.62 ml (3.54 mmol) of N,N-diisopropylethylamine were added and the mixture was stirred at room temperature for 20 min. 256 mg (0.92 mmol) of ent-benzyl (1-amino-4,4-difluoro-2-methylbutan-2-yl)carbamate (enantiomer A) from Example 27A were then added. After 60 min, water was added and the resulting solid was filtered off and washed with water. The residue was purified by preparative HPLC (RP18 column, mobile phase: acetonitrile/water gradient with addition of 0.1% TFA). The product fractions were combined and concentrated. 439 mg of the title compound were obtained (86% of theory).

LC-MS (Method 2): Rₜ=1.22 min

MS (ESpos): m/z=607 (M-TFA+H)⁻

¹H-NMR (400 MHz, DMSO-d₆): δ [ppm]=1.29 (s, 3H), 2.05-2.25 (m, 2H), 2.53 (s, 3H; overlapped with solvent peak), 3.54-3.62 (m, 2H; overlapped with solvent peak), 5.01 (s, 2H), 5.38 (s, 2H), 5.98-6.29 (m, 1H), 7.18-7.39 (m, 9H), 7.54-7.64 (m, 1H), 7.95 (t, 1H), 8.74 (d, 1H).

### Example 34A

### ent-Benzyl {1-[({6-chloro-8-[(2,6-difluorobenzyl)oxy]-2-methylimidazo[1,2-a]pyridin-3-yl}carbonyl)amino]-4,4-difluoro-2-methylbutan-2-yl}carbamate trifluoroacetate (enantiomer B)

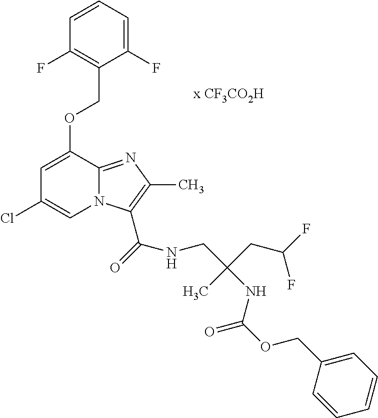 [SMILES: Cc1nc2c(OCc3c(F)cccc3F)cc(Cl)cn2c1C(=O)NCC(C)(CC(F)F)NC(=O)OCc1ccccc1]

250 mg (0.71 mmol) of 6-chloro-8-[(2,6-difluorobenzyl)oxy]-2-methylimidazo[1,2-a]pyridine-3-carboxylic acid from Example 5A were dissolved in 2.36 ml of DMF, 323 mg (0.85 mmol) of HATU and 0.62 ml (3.54 mmol) of N,N-diisopropylethylamine were added and the mixture was stirred at room temperature for 20 min. 246 mg (0.85 mmol, 93% purity) of ent-benzyl (1-amino-4,4-difluoro-2-methylbutan-2-yl)carbamate (enantiomer B) from Example 28A were then added. After 30 min, water was added and the resulting solid was filtered off and washed with water. The residue was purified by preparative HPLC (RP 18 column, mobile phase: acetonitrile/water gradient with addition of 0.1% TFA). The product fractions were combined and concentrated. 431 mg of the title compound were obtained (82% of theory).

LC-MS (Method 2): Rₜ=1.21 min

MS (ESpos): m/z=607 (M-TFA+H)⁻

¹H-NMR (500 MHz, DMSO-d₆): δ [ppm]=1.29 (s, 3H), 2.06-2.24 (m, 2H), 2.53 (s, 3H; overlapped with solvent peak), 3.55-3.62 (m, 2H), 5.01 (s, 2H), 5.38 (s, 2H), 6.00-6.29 (m, 1H), 7.19-7.39 (m, 9H), 7.55-7.64 (m, 1H), 7.99 (t, 1H), 8.74 (d, 1H).

### Example 35A

### ent-Benzyl {1-[({8-[(2,6-difluorobenzyl)oxy]-2-methylimidazo[1,2-a]pyridin-3-yl}carbonyl)amino]-5,5,5-trifluoro-2-methylpentan-2-yl}carbamate trifluoroacetate (enantiomer A)

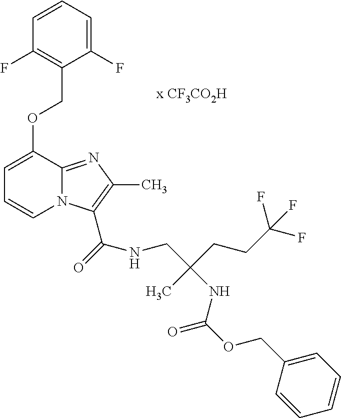 [SMILES: Cc1nc2c(OCc3c(F)cccc3F)cccn2c1C(=O)NCC(C)(CCC(F)(F)F)NC(=O)OCc1ccccc1]

80 mg (0.24 mmol) of 8-[(2,6-difluorobenzyl)oxy]-2-methylimidazo[1,2-a]pyridine-3-carboxylic acid from Example 8A were dissolved in 0.8 ml of DMF, 121 mg (0.32 mmol) of HATU and 0.21 ml (1.22 mmol) of N,N-diisopropylethylamine were added and the mixture was stirred at room temperature for 20 min. 102 mg (0.32 mmol, 95% purity) of ent-benzyl (1-amino-5,5,5-trifluoro-2-methylpentan-2-yl)carbamate (enantiomer A) were then added. After stirring at RT overnight, the mixture was concentrated, water, acetonitrile and TFA were added and the product was purified by preparative HPLC (RP 18 column, mobile phase: acetonitrile/water gradient with addition of 0.1% TFA). The product fractions were combined and concentrated. This gave 85 mg of the title compound (43% of theory; purity 88%).

LC-MS (Method 2): Rₜ=1.07 min

MS (ESpos): m/z=605 (M-TFA+H)⁻

### Example 36A

### ent-Benzyl {1-[({8-[(2,6-difluorobenzyl)oxy]-2-methylimidazo[1,2-a]pyridin-3-yl}carbonyl)amino]-5,5,5-trifluoro-2-methylpentan-2-yl}carbamate (enantiomer B)

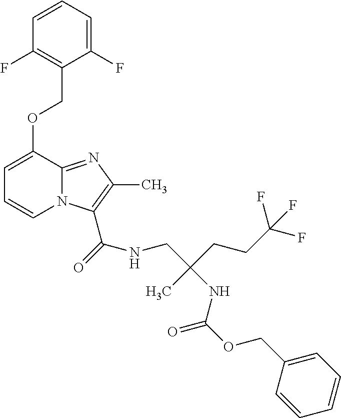 [SMILES: Cc1nc2c(OCc3c(F)cccc3F)cccn2c1C(=O)NCC(C)(CCC(F)(F)F)NC(=O)OCc1ccccc1]

80 mg (0.24 mmol) of 8-[(2,6-difluorobenzyl)oxy]-2-methylimidazo[1,2-a]pyridine-3-carboxylic acid from Example 8A were dissolved in 0.8 ml of DMF, 121 mg (0.32 mmol) of HATU and 0.21 ml (1.22 mmol) of N,N-diisopropylethylamine were added and the mixture was stirred at room temperature for 20 min. Subsequently, 102 mg (0.32 mmol, 95% purity) of ent-benzyl (1-amino-5,5,5-trifluoro-2-methylpentan-2-yl)carbamate (enantiomer B) from Example 14A were added. After 30 min, water was added and the resulting solid was filtered off and washed with water. The solid was dried under high vacuum. 150 mg of the title compound were obtained (99% of theory).

LC-MS (Method 2): Rₜ=1.08 min

MS (ESpos): m/z=605 (M+H)⁺

### Example 37A

### 3-[(2,3,6-Trifluorobenzyl)oxy]pyridine-2-amine

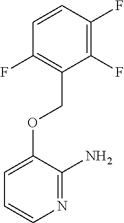 [SMILES: Nc1ncccc1OCc1c(F)ccc(F)c1F]

At RT, 29.05 g (537.7 mmol) of sodium methoxide were initially charged in 560 ml of methanol, 56.4 g (512.1 mmol) of 2-aminopyridin-3-ol were added and stirring at RT was continued for 15 min. The reaction mixture was concentrated under reduced pressure, the residue was taken up in 1400 ml of DMSO and 121 g (537.7 mmol) of 2-(bromomethyl)-1,3,4-trifluorobenzene were added. After 4 h at RT, the reaction mixture was poured onto 20 litres of water, the mixture was stirred for a further 15 min and the solid was filtered off. The solid was washed with 1 l of water and then purified by silica gel chromatography (mobile phase: cyclohexane/ethyl acetate=2/1). This gave 77.7 g of the title compound (60% of theory).

LC-MS (Method 2): Rₜ=0.48 min

MS (ESpos): m/z=255 (M+H)⁺

### Example 38A

### 5-Bromo-3-[(2,3,6-trifluorobenzyl)oxy]pyridine-2-amine

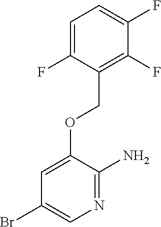 [SMILES: Nc1ncc(Br)cc1OCc1c(F)ccc(F)c1F]

76.4 g (300.5 mmol) of 3-[(2,3,6-trifluorobenzyl)oxy]pyridine-2-amine from Example 37A were suspended in 1300 ml of 10% strength sulphuric acid, and the mixture was cooled to 0° C. 18.6 ml (360.6 mmol) of bromine were dissolved in 200 ml of acetic acid and then, over 90 min, added dropwise to the reaction solution, cooled with ice. After the addition had ended, the mixture was stirred at 0° C. for a further 1.5 h and then diluted with 600 ml of ethyl acetate and stirred for 5 min, and the aqueous phase was separated off. The aqueous phase was extracted with ethyl acetate. The organic phases were combined and washed twice with saturated aqueous sodium bicarbonate solution and saturated sodium chloride solution. A mixture of dichloromethane/methanol (9/1) was added and the phases were separated. The organic phase was dried and concentrated. The residue was purified by preparative HPLC (RP18 column, Chromatorex C18, 10 μM, 350×100 mm, mobile phase: methanol/water gradient). This gave 44 g (44% of theory) of the title compound.

LC-MS (Method 2): Rₜ=0.97 min

MS (ESpos): m/z=333/335 (M+H)⁺

### Example 39A

### Ethyl 6-bromo-2-methyl-8-[(2,3,6-trifluorobenzyl)oxy]imidazo[1,2-a]pyridine-3-carboxylate

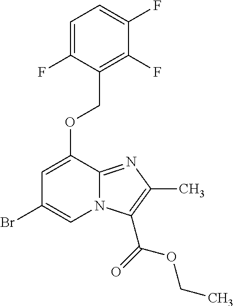 [SMILES: CCOC(=O)c1c(C)nc2c(OCc3c(F)ccc(F)c3F)cc(Br)cn12]

44 g (132.1 mmol) of 5-bromo-3-[(2,3,6-trifluorobenzyl)oxy]pyridine-2-amine from Example 38A was initially charged in 600 ml of ethanol, 25 g of powdered molecular sieve 3 Å and 108.7 g (660.4 mmol) of ethyl 2-chloroacetoacetate were added and the mixture was heated at reflux for 2 days. The reaction mixture was filtered and concentrated. The residue was suspended in dichloromethane and purified by silica gel chromatography (mobile phase: dichloromethane, dichloromethane/methanol=20/1). The product fractions were concentrated, 600 ml of acetonitrile were added to the residue, the mixture was stirred for 30 min and the solid was filtered off and dried. This gave 24.40 g (42% of theory) of the title compound.

LC-MS (Method 2): Rₜ=1.27 min

MS (ESpos): m/z=443/445 (M+H)⁺

¹H-NMR (400 MHz, DMSO-d₆): δ=1.36 (t, 3H), 2.54 (s, 3H; overlapped with DMSO signal), 4.37 (q, 2H), 5.41 (s, 2H), 7.26-7.36 (m, 1H), 7.42-7.46 (m, 1H), 7.64-7.75 (m, 1H), 9.00-9.03 (m, 1H).

### Example 40A

### 6-Bromo-2-methyl-8-[(2,3,6-trifluorobenzyl)oxy]imidazo[1,2-a]pyridine-3-carboxylic acid

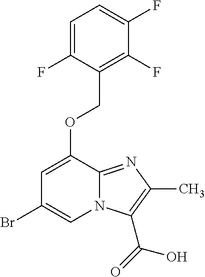 [SMILES: Cc1nc2c(OCc3c(F)ccc(F)c3F)cc(Br)cn2c1C(=O)O]

0.50 g (1.13 mmol) of ethyl 6-bromo-2-methyl-8-[(2,3,6-trifluorobenzyl)oxy]imidazo[1,2-a]pyridine-3-carboxylate from Example 39A were dissolved in 24 ml of THF/methanol (5/1), 5.6 ml (5.6 mmol) of lithium hydroxide solution (1 M in water) were added and the mixture was stirred at 40° C. overnight. The mixture was concentrated, the residue was suspended in 24 ml of dioxane and 5.6 ml (5.6 mmol) of a 1 N aqueous sodium hydroxide solution were added. The reaction mixture was stirred at RT overnight. The reaction solution was concentrated almost completely. The residue was taken up in a little THF/water and acidified with hydrochloric acid. The solid formed was stirred at room temperature for 30 min and then filtered off and washed with water. The solid was dried under high vacuum. This gave 0.44 g of the title compound (94% of theory).

LC-MS (Method 2): Rₜ=0.90 min

MS (ESpos): m/z=415/417 (M+H)⁺

¹H-NMR (400 MHz, DMSO-d₆): δ=2.54 (s, 3H; overlapped by DMSO signal), 5.41 (s, 2H), 7.26-7.35 (m, 1H), 7.38-7.41 (m, 1H), 7.63-7.74 (m, 1H), 9.05-9.09 (m, 1H), 13.34 (br. s, 1H).

### Example 41A

### ent-Benzyl {1-[({6-bromo-2-methyl-8-[(2,3,6-trifluorobenzyl)oxy]imidazo[1,2-a]pyridin-3-yl}carbonyl)amino]-5,5,5-trifluoro-2-methylpentan-2-yl}carbamate trifluoroacetate (enantiomer B)

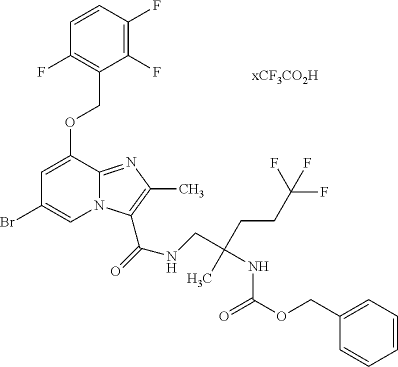 [SMILES: Cc1nc2c(OCc3c(F)ccc(F)c3F)cc(Br)cn2c1C(=O)NCC(C)(CCC(F)(F)F)NC(=O)OCc1ccccc1]

210 mg (0.50 mmol) of 6-bromo-2-methyl-8-[(2,3,6-trifluorobenzyl)oxy]imidazo[1,2-a]pyridine-3-carboxylic acid from Example 40A were dissolved in 1.75 ml of DMF, 245 mg (0.64 mmol) of HATU and 0.43 ml (2.48 mmol) of N,N-diisopropylethylamine were added and the mixture was stirred at room temperature for 10 min. Subsequently, 206 mg (0.64 mmol, 95% purity) of ent-benzyl (1-amino-5,5,5-trifluoro-2-methylpentan-2-yl)carbamate (enantiomer B) from Example 14A were added. After all the starting material had reacted (about 60 min), acetonitrile/water and TFA was added and the reaction solution was then purified by preparative HPLC (RP 18 column, mobile phase: acetonitrile/water gradient with addition of 0.1% TFA). This gave 294 mg of the title compound (68% of theory, purity 94%).

LC-MS (Method 2): Rₜ=1.34 min

MS (ESpos): m/z=701/703 (M+H)+

### WORKING EXAMPLES

### Example 1

### ent-N-(2-amino-5-fluoro-2-methylpentyl)-8-[(2,6-difluorobenzyl)oxy]-2-methylimidazo[1,2-a]pyridine-3-carboxamide (enantiomer A)

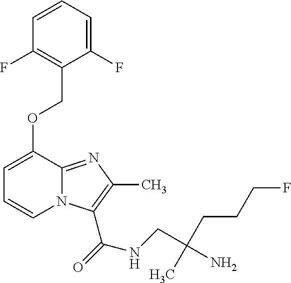 [SMILES: Cc1nc2c(OCc3c(F)cccc3F)cccn2c1C(=O)NCC(C)(N)CCCF]

397 mg (0.50 mmol, purity 91%) of ent-benzyl {1-[({6-chloro-8-[(2,6-difluorobenzyl)oxy]-2-methylimidazo[1,2-a]pyridin-3-yl}carbonyl)amino]-5-fluoro-2-methylpentan-2-yl}carbamate trifluoroacetate (enantiomer A) from Example 31A were dissolved in 12.9 ml of ethanol, 16 mg of palladium on activated carbon (10%) were added and the mixture was hydrogenated at standard pressure for 1.5 hours. The reaction solution was filtered by means of a Millipore filter and the filtrate was concentrated. The residue was purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with a little saturated aqueous sodium hydrogencarbonate solution. The aqueous phase was extracted twice with dichloromethane The combined organic phases were dried over sodium sulphate, filtered, concentrated and lyophilized. This gave 73 mg of the target compound (33% of theory).

LC-MS (Method 2): Rₜ=0.56 min

MS (ESpos): m/z=435 (M+H)⁺

¹H-NMR (400 MHz, DMSO-d₆): δ [ppm]=1.02 (s, 3H), 1.30-1.42 (m, 2H), 1.43-1.88 (m, 4H), 2.53 (s, 3H; overlapped by solvent peak), 3.17-3.30 (m, 2H), 4.30-4.39 (m, 1H), 4.42-4.52 (m, 1H), 5.30 (s, 2H), 6.92 (t, 1H), 7.00 (d, 1H), 7.18-7.28 (m, 2H), 7.54-7.63 (m, 1H), 7.65-7.77 (m, 1H), 8.62 (d, 1H).

### Example 2

### ent-N-(2-amino-5-fluoro-2-methylpentyl)-8-[(2,6-difluorobenzyl)oxy]-2-methylimidazo[1,2-a]pyridine-3-carboxamide (enantiomer B)

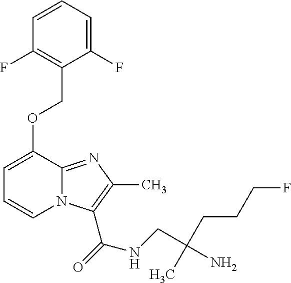 [SMILES: Cc1nc2c(OCc3c(F)cccc3F)cccn2c1C(=O)NCC(C)(N)CCCF]

328 mg (0.43 mmol, purity 94%) of ent-benzyl {1-[({6-chloro-8-[(2,6-difluorobenzyl)oxy]-2-methylimidazo[1,2-a]pyridin-3-yl}carbonyl)amino]-5-fluoro-2-methylpentan-2-yl}carbamate trifluoroacetate (enantiomer B) from Example 32A were dissolved in 11.1 ml of ethanol, 14 mg of palladium on activated carbon (10%) were added and the mixture was hydrogenated at standard pressure for 3 hours. The reaction solution was filtered through Celite, the filter cake was washed with ethanol and the filtrate was concentrated. The residue was purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with a little saturated aqueous sodium hydrogencarbonate solution. The aqueous phase was extracted twice with dichloromethane. The combined organic phases were dried over sodium sulphate, filtered, concentrated and lyophilized. This gave 53 mg of the target compound (28% of theory).

LC-MS (Method 4): Rₜ=2.11 min

MS (ESpos): m/z=435 (M+H)⁺

¹H-NMR (500 MHz, DMSO-d₆): δ [ppm]=1.02 (s, 3H), 1.32-1.42 (m, 2H), 1.52 (br. s, 2H), 1.62-1.86 (m, 4H), 2.53 (s, 3H; overlapped with solvent peak), 3.17-3.29 (m, 2H), 4.32-4.39 (m, 1H), 4.43-4.50 (m, 1H), 5.30 (s, 2H), 6.92 (t, 1H), 7.00 (d, 1H), 7.18-7.27 (m, 2H), 7.55-7.63 (m, 1H), 7.66-7.75 (m, 1H), 8.62 (d, 1H).

### Example 3

### ent-N-(2-amino-4,4-difluoro-2-methylbutyl)-8-[(2,6-difluorobenzyl)oxy]-2-methylimidazo[1,2-a]pyridine-3-carboxamide (enantiomer A)

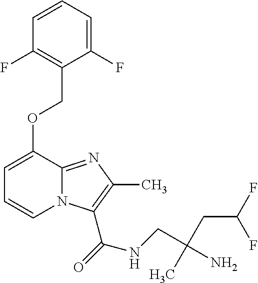 [SMILES: Cc1nc2c(OCc3c(F)cccc3F)cccn2c1C(=O)NCC(C)(N)CC(F)F]

439 mg (0.61 mmol) of ent-benzyl {1-[({6-chloro-8-[(2,6-difluorobenzyl)oxy]-2-methylimidazo[1,2-a]pyridin-3-yl}carbonyl)amino]-4,4-difluoro-2-methylbutan-2-yl}carbamate trifluoroacetate (enantiomer A) from Example 33A were dissolved in 15.6 ml of ethanol, 19 mg of palladium on activated carbon (10%) were added and the mixture was hydrogenated at standard pressure for 75 min. The reaction solution was filtered through a Millipore filter and the filtrate was concentrated. The residue was purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with a little saturated aqueous sodium hydrogencarbonate solution. The aqueous phase was extracted twice with dichloromethane. The combined organic phases were dried over sodium sulphate, filtered, concentrated and lyophilized. This gave 85 mg of the target compound (31% of theory).

LC-MS (Method 2): Rₜ=0.60 min

MS (ESpos): m/z=439 (M+H)⁺

¹H-NMR (400 MHz, DMSO-d₆): δ [ppm]=1.08 (s, 3H), 1.70 (br. s, 2H), 1.83-1.99 (m, 2H), 2.54 (s, 3H; overlapped with solvent peak), 3.20-3.35 (m, 2H; overlapped with solvent peak), 5.30 (s, 2H), 6.08-6.42 (m, 1H), 6.92 (t, 1H), 7.00 (d, 1H), 7.18-7.28 (m, 2H), 7.54-7.64 (m, 1H), 7.87 (t, 1H), 8.62 (d, 1H).

### Example 4

### ent-N-(2-amino-4,4-difluoro-2-methylbutyl)-8-[(2,6-difluorobenzyl)oxy]-2-methylimidazo[1,2-a]pyridine-3-carboxamide (enantiomer B)

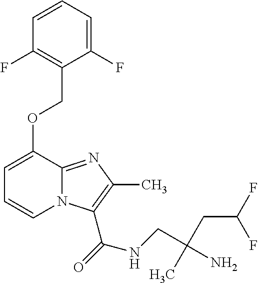 [SMILES: Cc1nc2c(OCc3c(F)cccc3F)cccn2c1C(=O)NCC(C)(N)CC(F)F]

431 mg (0.59 mmol) of ent-benzyl {1-[({6-chloro-8-[(2,6-difluorobenzyl)oxy]-2-methylimidazo[1,2-a]pyridin-3-yl}carbonyl)amino]-4,4-difluoro-2-methylbutan-2-yl}carbamate trifluoroacetate (enantiomer B) from Example 34A were dissolved in 15.1 ml of ethanol, 19 mg of palladium on activated carbon (10%) were added and the mixture was hydrogenated at standard pressure for 75 min. The reaction solution was filtered through a Millipore filter and the filtrate was concentrated. The residue was purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with a little saturated aqueous sodium hydrogencarbonate solution. The aqueous phase was extracted twice with dichloromethane. The combined organic phases were dried over sodium sulphate, filtered, concentrated and lyophilized. This gave 90 mg of the target compound (34% of theory).

LC-MS (Method 2): Rₜ=0.60 min

MS (ESpos): m/z=439 (M+H)⁺

¹H-NMR (500 MHz, DMSO-d₆): δ [ppm]=1.08 (s, 3H), 1.70 (br. s, 2H), 1.84-1.98 (m, 2H), 2.54 (s, 3H; overlapped with solvent peak), 3.22-3.32 (m, 2H; overlapped with solvent peak), 5.30 (s, 2H), 6.10-6.38 (m, 1H), 6.92 (t, 1H), 7.00 (d, 1H), 7.18-7.27 (m, 2H), 7.55-7.63 (m, 1H), 7.88 (t, 1H), 8.62 (d, 1H).

### Example 5

### ent-N-(2-amino-5,5,5-trifluoro-2-methylpentyl)-8-[(2,6-difluorobenzyl)oxy]-2-methylimidazo[1,2-a]pyridine-3-carboxamide (enantiomer A)

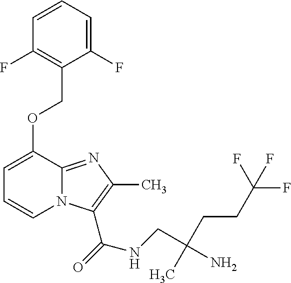 [SMILES: Cc1nc2c(OCc3c(F)cccc3F)cccn2c1C(=O)NCC(C)(N)CCC(F)(F)F]

85 mg (0.11 mmol, purity 88%) of ent-benzyl {1-[({8-[(2,6-difluorobenzyl)oxy]-2-methylimidazo[1,2-a]pyridin-3-yl}carbonyl)amino]-5,5,5-trifluoro-2-methylpentan-2-yl}carbamate trifluoroacetate (enantiomer A) from Example 35A were dissolved in 2.7 ml of ethanol, 3.5 mg of palladium on activated carbon (10%) were added and the mixture was hydrogenated at standard pressure for 1.5 hours. The reaction solution was filtered through a Millipore filter, the filter cake was washed with ethanol and the filtrate was concentrated. The residue was purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with a little saturated aqueous sodium hydrogencarbonate solution. The aqueous phase was extracted twice with dichloromethane. The combined organic phases were dried over sodium sulphate, filtered, concentrated and lyophilized. This gave 95 mg of the target compound (95% of theory, purity 93%).

LC-MS (Method 2): Rₜ=0.63 min

MS (ESpos): m/z=471 (M+H)⁺

¹H-NMR (400 MHz, DMSO-d₆): δ [ppm]=1.02 (s, 3H), 1.47-1.57 (m, 2H), 1.61 (br. s, 2H), 2.24-2.48 (m, 2H), 2.55 (s, 3H; overlapped with solvent peak), 3.18-3.31 (m, 2H, partially overlapped with solvent peak), 5.31 (s, 2H), 6.93 (t, 1H), 7.01 (d, 1H), 7.18-7.27 (m, 2H), 7.55-7.64 (m, 1H), 7.76-7.83 (m, 1H), 8.59 (d, 1H).

### Example 6

### ent-N-(2-amino-5,5,5-trifluoro-2-methylpentyl)-8-[(2,6-difluorobenzyl)oxy]-2-methylimidazo[1,2-a]pyridine-3-carboxamide (enantiomer B)

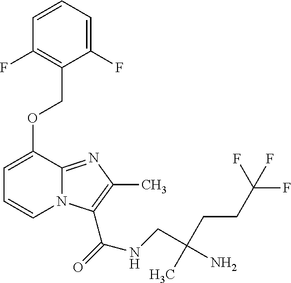 [SMILES: Cc1nc2c(OCc3c(F)cccc3F)cccn2c1C(=O)NCC(C)(N)CCC(F)(F)F]

150 mg (0.21 mmol) of ent-benzyl {1-[({8-[(2,6-difluorobenzyl)oxy]-2-methylimidazo[1,2-a]pyridin-3-yl}carbonyl)amino]-5,5,5-trifluoro-2-methylpentan-2-yl}carbamate (enantiomer B) from Example 36A were dissolved in 5.2 ml of ethanol, 32 (0.42 mmol) of TFA and 7 mg of palladium on activated carbon (10%) were added and the mixture was hydrogenated at standard pressure for 5.5 hours. The reaction solution was filtered through a Millipore filter, the filter cake was washed with ethanol and the filtrate was concentrated. The residue was purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with a little saturated aqueous sodium hydrogencarbonate solution. The aqueous phase was extracted twice with dichloromethane. The combined organic phases were dried over sodium sulphate, filtered, concentrated and lyophilized. This gave 95 mg of the target compound (98% of theory).

LC-MS (Method 2): Rₜ=0.66 min

MS (ESpos): m/z=471 (M+H)⁺

¹H-NMR (400 MHz, DMSO-d₆): δ [ppm]=1.03 (s, 3H), 1.47-1.58 (m, 2H), 1.69 (br. s, 2H), 2.25-2.48 (m, 2H), 2.55 (s, 3H; overlapped with solvent peak), 3.18-3.31 (m, 2H, partially overlapped with solvent peak), 5.31 (s, 2H), 6.93 (t, 1H), 7.01 (d, 1H), 7.18-7.27 (m, 2H), 7.55-7.64 (m, 1H), 7.77-7.83 (m, 1H), 8.60 (d, 1H).

### Example 7

### ent-N-(2-amino-5,5,5-trifluoro-2-methylpentyl)-2-methyl-8-[(2,3,6-trifluorobenzyl)oxy]imidazo[1,2-a]pyridine-3-carboxamide (enantiomer B)

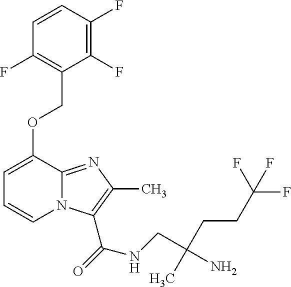 [SMILES: Cc1nc2c(OCc3c(F)ccc(F)c3F)cccn2c1C(=O)NCC(C)(N)CCC(F)(F)F]

294 mg (0.34 mmol, purity 94%) of ent-benzyl {1-[({6-bromo-2-methyl-8-[(2,3,6-trifluorobenzyl)oxy]imidazo[1,2-a]pyridin-3-yl}carbonyl)amino]-5,5,5-trifluoro-2-methylpentan-2-yl}carbamate trifluoroacetate (enantiomer B) from Example 41A were dissolved in 36 ml of ethanol, 78 μl (1.02 mmol) of TFA and 11 mg of palladium on activated carbon (10%) were added, and hydrogenation was carried out at standard pressure for 6 hours. The reaction solution was filtered through a Millipore filter, the filter cake was washed with ethanol and the filtrate was concentrated on a rotary evaporator. The residue was purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with a little saturated aqueous sodium hydrogencarbonate solution. The aqueous phase was re-extracted twice with dichloromethane. The combined organic phases were dried over sodium sulphate, filtered, concentrated and lyophilized. This gave 138 mg of the target compound (82% of theory, purity 98%).

LC-MS (Method 2): Rₜ=0.64 min

MS (ESpos): m/z=489 (M+H)⁺

¹H-NMR (400 MHz, DMSO-d₆): δ [ppm]=1.02 (s, 3H), 1.47-1.70 (m, 4H), 2.21-2.48 (m, 2H), 2.56 (s, 3H), 3.18-3.30 (m, 2H, partially overlapped with solvent peak), 5.36 (s, 2H), 6.93 (t, 1H), 7.01 (d, 1H), 7.24-7.33 (m, 1H), 7.59-7.72 (m, 1H), 7.76-7.84 (m, 1H), 8.60 (d, 1H).

### B. ASSESSMENT OF PHARMACOLOGICAL EFFICACY

The following abbreviations are used:

ATP adenosine triphosphate

Brij35 polyoxyethylene(23) lauryl ether

BSA bovine serum albumin

DTT dithiothreitol

TEA triethanolamine

The pharmacological action of the compounds of the invention can be demonstrated in the following assays:

B-1. Measurement of sGC Enzyme Activity by Means of PPi Detection

Soluble guanylyl cyclase (sGC) converts GTP to cGMP and pyrophosphate (PPi) when stimulated. PPi is detected with the aid of the method described in WO 2008/061626. The signal that arises in the assay increases as the reaction progresses and serves as a measure of the sGC enzyme activity. With the aid of a PPi reference curve, the enzyme can be characterized in a known manner, for example in terms of conversion rate, stimulability or Michaelis constant.

Practice of the Test

To conduct the test, 29 μl of enzyme solution (0-10 nM soluble guanylyl cyclase (prepared according to H\ddot{n}icka et al., Journal of Molecular Medicine 77(1999)14-23), in 50 mM TEA, 2 mM magnesium chloride, 0.1% BSA (fraction V), 0.005% Brij 35, pH 7.5) were initially charged in the microplate, and 1 μl of the stimulator solution (0-10 μM 3-morpholinosydnonimine, SIN-1, Merck in DMSO) was added. The microplate was incubated at RT for 10 min. Then 20 μl of detection mix (1.2 nM Firefly Luciferase (Photinus pyralis luciferase, Promega), 29 μM dehydroluciferin (prepared according to Bitler & McElroy, Arch. Biochem. Biophys. 72 (1957) 358), 122 μM luciferin (Promega), 153 μM ATP (Sigma) and 0.4 mM DTT (Sigma) in 50 mM TEA, 2 mM magnesium chloride, 0.1% BSA (fraction V), 0.005% Brij 35, pH 7.5) were added. The enzyme reaction was started by adding 20 μl of substrate solution (1.25 mM guanosine 5′-triphosphate (Sigma) in 50 mM TEA, 2 mM magnesium chloride, 0.1% BSA (fraction V), 0.005% Brij, pH 7.5) and analysed continuously in a luminometer.

B-2. Effect on a Recombinant Guanylate Cyclase Reporter Cell Line

The cellular activity of the compounds according to the invention is determined using a recombinant guanylate cyclase reporter cell line, as described in F. Wunder et al., Anal. Biochem. 339, 104-112 (2005).

Representative MEC values (MEC=minimum effective concentration) for the compounds of the invention are shown in the table below (in some cases as mean values for individual determinations):

TABLE A

| Example | MEC [μM] |
|---|---|
| 1 | 0.3 |
| 2 | 0.3 |
| 3 | 0.3 |
| 4 | 0.3 |
| 5 | 0.1 |
| 6 | 0.03 |
| 7 | 0.09 |

B-3. Vasorelaxant Effect in Vitro

Rabbits are stunned by a blow to the neck and exsanguinated. The aorta is removed, freed from adhering tissue and divided into rings of width 1.5 mm, which are placed individually under prestress into 5 ml organ baths with carbogen-sparged Krebs-Henseleit solution at 37° C. having the following composition (each in mM): sodium chloride: 119; potassium chloride: 4.8; calcium chloride dihydrate: 1; magnesium sulphate heptahydrate: 1.4; potassium dihydrogenphosphate: 1.2; sodium bicarbonate: 25; glucose: 10. The contractile force is determined with Statham UC2 cells, amplified and digitalized using A/D transducers (DAS-1802 HC, Keithley Instruments Munich), and recorded in parallel on linear recorders. To obtain a contraction, phenylephrine is added to the bath cumulatively in increasing concentration. After several control cycles, the substance to be studied is added in increasing dosage each time in every further run, and the magnitude of the contraction is compared with the magnitude of the contraction attained in the last preceding run. This is used to calculate the concentration needed to reduce the magnitude of the control value by 50% (IC₅₀ value). The standard administration volume is 5 μl; the DMSO content in the bath solution corresponds to 0.1%.

B-4. Blood Pressure Measurement on Anaesthetized Rats

Male Wistar rats having a body weight of 300-350 g are anaesthetized with thiopental (100 mg/kg i.p.). After tracheotomy, a catheter is introduced into the femoral artery to measure the blood pressure. The substances to be tested are administered as solutions, either orally by means of a gavage or intravenously via the femoral vein (Stasch et al. Br. J. Pharmacol. 2002; 135: 344-355).

B-5. Radiotelemetry Measurement of Blood Pressure in Conscious, Spontaneously Hypertensive Rats

A commercially available telemetry system from DATA SCIENCES INTERNATIONAL DSI, USA, is employed for the blood pressure measurement on conscious rats described below.

The system consists of 3 main components:

implantable transmitters (Physiotel® telemetry transmitter)

receivers (Physiotel® receiver) which are linked via a multiplexer (DSI Data Exchange Matrix) to a

data acquisition computer.

The telemetry system makes it possible to continuously record blood pressure, heart rate and body motion of conscious animals in their usual habitat.

Animal Material

The studies are conducted on adult female spontaneously hypertensive rats (SHR Okamoto) with a body weight of >200 g. SHR/NCrl from the Okamoto Kyoto School of Medicine, 1963, were a cross of male Wistar Kyoto rats having greatly elevated blood pressure and female rats having slightly elevated blood pressure, and were handed over at F13 to the U.S. National Institutes of Health.

After transmitter implantation, the experimental animals are housed singly in type 3 Makrolon cages. They have free access to standard feed and water.

The day/night rhythm in the experimental laboratory is changed by the room lighting at 6:00 am and at 7:00 pm.

Transmitter Implantation

The TA11 PA-C40 telemetry transmitters used are surgically implanted under aseptic conditions in the experimental animals at least 14 days before the first experimental use. The animals instrumented in this way can be used repeatedly after the wound has healed and the implant has settled.

For the implantation, the fasted animals are anaesthetized with pentobarbital (Nembutal, Sanofi: 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD™, 3M). The transmitter housing is fixed intraperitoneally to the abdominal wall muscle, and the wound is closed layer by layer.

An antibiotic (Tardomyocel COMP, Bayer, 1 ml/kg s.c.) is administered postoperatively for prophylaxis of infection.

Substances and Solutions

Unless stated otherwise, the substances to be studied are administered orally by gavage to a group of animals in each case (n=6). In accordance with an administration volume of 5 ml/kg of body weight, the test substances are dissolved in suitable solvent mixtures or suspended in 0.5% tylose.

A solvent-treated group of animals is used as control.

Experimental Outline

The telemetry measuring unit present is configured for 24 animals. Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch. They are switched to transmission in the run-up to the experiment. The signals emitted can be detected online by a data acquisition system (Dataquest™ A.R.T. for WINDOWS, DSI) and processed accordingly. The data are stored in each case in a file created for this purpose and bearing the experiment number.

In the standard procedure, the following are measured for 10-second periods in each case:

systolic blood pressure (SBP)

diastolic blood pressure (DBP)

mean arterial pressure (MAP)

heart rate (HR)

activity (ACT).

The acquisition of measurements is repeated under computer control at 5-minute intervals. The source data obtained as absolute values are corrected in the diagram with the currently measured barometric pressure (Ambient Pressure Reference Monitor; APR-1) and stored as individual data. Further technical details are given in the extensive documentation from the manufacturer company (DSI).

Unless indicated otherwise, the test substances are administered at 9:00 am on the day of the experiment. Following the administration, the parameters described above are measured over 24 hours.

Evaluation

After the end of the experiment, the acquired individual data are sorted using the analysis software (DATAQUEST™ A.R.T.™ ANALYSIS). The blank value is assumed here to be the time 2 hours before administration, and so the selected data set encompasses the period from 7:00 am on the day of the experiment to 9:00 am on the following day.

The data are smoothed over a predefinable period by determination of the average (15-minute average) and transferred as a text file to a storage medium. The measured values presorted and compressed in this way are transferred to Excel templates and tabulated. For each day of the experiment, the data obtained are stored in a dedicated file bearing the number of the experiment. Results and test protocols are stored in files in paper form sorted by numbers.

Literature:

Klaus Witte, Kai Hu, Johanna Swiatek, Claudia Müssig, Georg Ertl and Björn Lemmer: Experimental heart failure in rats: effects on cardiovascular circadian rhythms and on myocardial β-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994.

B-6. Determination of Pharmacokinetic Parameters Following Intravenous and Oral Administration

The pharmacokinetic parameters of the compounds according to the invention are determined in male CD-1 mice, male Wistar rats and female beagles. Intravenous administration in the case of mice and rats is effected by means of a species-specific plasma/DMSO formulation, and in the case of dogs by means of a water/PEG400/ethanol formulation. In all species, oral administration of the dissolved substance is performed via gavage, based on a water/PEG400/ethanol formulation. The removal of blood from rats is simplified by inserting a silicone catheter into the right Vena jugularis externa prior to substance administration. The operation is carried out at least one day prior to the experiment with isofluran anaesthesia and administration of an analgesic (atropine/rimadyl (3/1) 0.1 ml s.c.). The blood is taken (generally more than 10 time points) within a time window including terminal time points of at least 24 to a maximum of 72 hours after substance administration. The blood is removed into heparinized tubes. The blood plasma is then obtained by centrifugation; if required, it can be stored at −20° C. until further processing.

An internal standard (which may also be a chemically unrelated substance) is added to the samples of the compounds of the invention, calibration samples and qualifiers, and there follows protein precipitation by means of acetonitrile in excess. Addition of a buffer solution matched to the LC conditions, and subsequent vortexing, is followed by centrifugation at 1000 g. The supernatant is analysed by LC-MS/MS using C18 reversed-phase columns and variable mobile phase mixtures. The substances are quantified via the peak heights or areas from extracted ion chromatograms of specific selected ion monitoring experiments.

The plasma concentration/time plots determined are used to calculate the pharmacokinetic parameters such as AUC, Cₘₐₓ, t_{1/2} (terminal half-life), F (bioavailability), MRT (mean residence time) and CL (clearance), by means of a validated pharmacokinetic calculation program.

Since the substance quantification is performed in plasma, it is necessary to determine the blood/plasma distribution of the substance in order to be able to adjust the pharmacokinetic parameters correspondingly. For this purpose, a defined amount of substance is incubated in heparinized whole blood of the species in question in a rocking roller mixer for 20 min. After centrifugation at 1000 g, the plasma concentration is measured (by means of LC-MS/MS; see above) and determined by calculating the ratio of the C_{blood}/Cₚₗₐₛₘₐ value.

B-7. Metabolic Study

To determine the metabolic profile of the inventive compounds, they are incubated with recombinant human cytochrome P450 (CYP) enzymes, liver microsomes or primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds of the invention were incubated with a concentration of about 0.1-10 μM. To this end, stock solutions of the compounds of the invention having a concentration of 0.01-1 mM in acetonitrile were prepared, and then pipetted with a 1:100 dilution into the incubation mixture. Liver microsomes and recombinant enzymes were incubated at 37° C. in 50 mM potassium phosphate buffer pH 7.4 with and without an NADPH-generating system consisting of 1 mM NADP⁺, 10 mM glucose-6-phosphate and 1 unit glucose-6-phosphate dehydrogenase. Primary hepatocytes were incubated in suspension in Williams E medium, likewise at 37° C. After an incubation time of 0-4 h, the incubation mixtures were stopped with acetonitrile (final concentration about 30%) and the protein was centrifuged off at about 15 000×g. The samples thus stopped were either analysed directly or stored at −20° C. until analysis.

The analysis is carried out by high-performance liquid chromatography with ultraviolet and mass spectrometry detection (HPLC-UV-MS/MS). To this end, the supernatants of the incubation samples are chromatographed with suitable C18 reversed-phase columns and variable mobile phase mixtures of acetonitrile and 10 mM aqueous ammonium formate solution or 0.05% formic acid. The UV chromatograms in conjunction with mass spectrometry data serve for identification, structural elucidation and quantitative estimation of the metabolites, and for quantitative metabolic reduction of the compound of the invention in the incubation mixtures.

B-8. Caco-2 Permeability Test

The permeability of a test substance was determined with the aid of the Caco-2 cell line, an established in vitro model for permeability prediction at the gastrointestinal barrier (Artursson, P. and Karlsson, J. (1991). Correlation between oral drug absorption in humans and apparent drug permeability coefficients in human intestinal epithelial (Caco-2) cells. Biochem. Biophys. 175 (3), 880-885). The Caco-2 cells (ACC No. 169, DSMZ, Deutsche Sammlung von Mikroorganismen and Zellkulturen, Braunschweig, Germany) were sown in 24-well plates having an insert and cultivated for 14 to 16 days. For the permeability studies, the test substance was dissolved in DMSO and diluted to the final test concentration with transport buffer (Hanks Buffered Salt Solution, Gibco/Invitrogen, with 19.9 mM glucose and 9.8 mM HEPES). In order to determine the apical to basolateral permeability (PₐₚₚA-B) of the test substance, the solution comprising the test substance was applied to the apical side of the Caco-2 cell monolayer, and transport buffer to the basolateral side. In order to determine the basolateral to apical permeability (PₐₚₚB-A) of the test substance, the solution comprising the test substance was applied to the basolateral side of the Caco-2 cell monolayer, and transport buffer to the apical side. At the start of the experiment, samples were taken from the respective donor compartment in order to ensure the mass balance. After an incubation time of two hours at 37° C., samples were taken from the two compartments. The samples were analysed by means of LC-MS/MS and the apparent permeability coefficients (Pₐₚₚ) were calculated. For each cell monolayer, the permeability of Lucifer Yellow was determined to ensure cell layer integrity. In each test run, the permeability of atenolol (marker for low permeability) and sulfasalazine (marker for active excretion) was also determined as quality control.

B-9. hERG Potassium Current Assay

The hERG (human ether-a-go-go related gene) potassium current makes a significant contribution to the repolarization of the human cardiac action potential (Scheel et al., 2011) Inhibition of this current by pharmaceuticals can in rare cases cause potentially lethal cardiac arrythmia, and is therefore studied at an early stage during drug development.

The functional hERG assay used here is based on a recombinant HEK293 cell line which stably expresses the KCNH2(HERG) gene (Zhou et al., 1998). These cells are studied by means of the “whole-cell voltage-clamp” technique (Hamill et al., 1981) in an automated system (Patchliner™; Nanion, Munich, Germany), which controls the membrane voltage and measures the hERG potassium current at room temperature. The PatchControlHT™ software (Nanion) controls the Patchliner system, data capture and data analysis. The voltage is controlled by 2 EPC-10 quadro amplifiers controlled by the PatchMasterPro™ software (both: HEKA Elektronik, Lambrecht, Germany). NPC-16 chips with moderate resistance (˜2 MΩ; Nanion) serve as the planar substrate for the voltage clamp experiments.

NPC-16 chips are filled with intra- and extracellular solution (cf. Himmel, 2007) and with cell suspension. After forming a gigaohm seal and establishing whole-cell mode (including several automated quality control steps), the cell membrane is clamped at the −80 mV holding potential. The subsequent voltage clamp protocol changes the command voltage to +20 mV (for 1000 ms), −120 mV (for 500 ms), and back to the −80 mV holding potential; this is repeated every 12 s. After an initial stabilization phase (about 5-6 minutes), test substance solution is introduced by pipette in rising concentrations (e.g. 0.1, 1, and 10 ∥mol/l) (exposure about 5-6 minutes per concentration), followed by several washing steps.

The amplitude of the inward “tail” current which is generated by a change in potential from +20 mV to −120 mV serves to quantify the hERG potassium current, and is described as a function of time (IgorPro™ Software). The current amplitude at the end of various time intervals (for example stabilization phase before test substance, first/second/third concentration of test substance) serves to establish a concentration/effect curve, from which the half-maximum inhibiting concentration IC₅₀ of the test substance is calculated.
Hamill O P, Marty A, Neher E, Sakmann B, Sigworth F J. Improved patch-clamp techniques for high-resolution current recording from cells and cell-free membrane patches. Pfluegers Arch 1981; 391:85-100.
Himmel H M. Suitability of commonly used excipients for electrophysiological in-vitro safety pharmacology assessment of effects on hERG potassium current and on rabbit Purkinje fiber action potential. J Pharmacol Toxicol Methods 2007; 56:145-158.
Scheel O, Himmel H, Rascher-Eggstein G, Knott T. Introduction of a modular automated voltage-clamp platform and its correlation with manual human ether-a-go-go related gene voltage-clamp data. Assay Drug Dev Technol 2011; 9:600-607.
Zhou Z F, Gong Q, Ye B, Fan Z, Makielski J C, Robertson G A, January C T. Properties of hERG channels stably expressed in HEK293 cells studied at physiological temperature. Biophys J 1998; 74:230-241.

### C. WORKING EXAMPLES OF PHARMACEUTICAL COMPOSITIONS

The compounds of the invention can be converted to pharmaceutical formulations as follows:

Tablet:

Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

Production:

The mixture of compound of the invention, lactose and starch is granulated with a 5% solution (w/w) of the PVP in water. The granules are dried and then mixed with the magnesium stearate for 5 minutes. This mixture is compressed using a conventional tabletting press (see above for format of the tablet). The guide value used for the pressing is a pressing force of 15 kN.

Suspension for Oral Administration:

Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

Production:

The Rhodigel is suspended in ethanol; the compound of the invention is added to the suspension. The water is added while stirring. The mixture is stirred for about 6 h until the swelling of the Rhodigel is complete.

Solution for Oral Administration:

Composition:

500 mg of the compound of the invention, 2.5 g of polysorbate and 97 g of polyethylene glycol 400. 20 g of oral solution correspond to a single dose of 100 mg of the compound of the invention.

Production:

The compound of the invention is suspended in the mixture of polyethylene glycol and polysorbate with stirring. The stirring operation is continued until dissolution of the compound of the invention is complete.

i.v. Solution:

The compound of the invention is dissolved in a concentration below the saturation solubility in a physiologically acceptable solvent (e.g. isotonic saline solution, glucose solution 5% and/or PEG 400 solution 30%). The resulting solution is subjected to sterile filtration and dispensed into sterile and pyrogen-free injection vessels.

1. Compound having the systematic name ent-N-(2-amino-5-fluoro-2-methylpentyl)-8-[(2,6-difluorobenzyl)oxy]-2-methylimidazo[1,2-a]pyridine-3-carboxamide (enantiomer A) and the structural formula 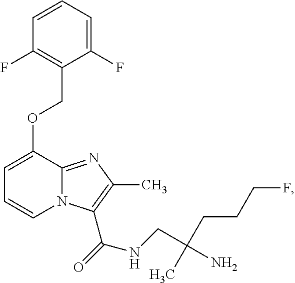 [SMILES: Cc1nc2c(OCc3c(F)cccc3F)cccn2c1C(=O)NCC(C)(N)CCCF]
and the N-oxides, salts, solvates, salts of the N-oxides and solvates of the N-oxides and salts thereof.

2. Compound having the systematic name ent-N-(2-amino-5-fluoro-2-methylpentyl)-8-[(2,6-difluorobenzyl)oxy]-2-methylimidazo[1,2-a]pyridine-3-carboxamide (enantiomer B) and the structural formula 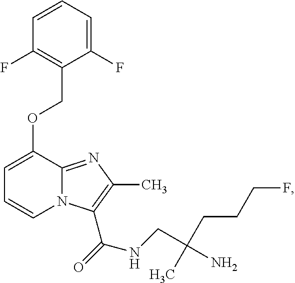 [SMILES: Cc1nc2c(OCc3c(F)cccc3F)cccn2c1C(=O)NCC(C)(N)CCCF]
and the N-oxides, salts, solvates, salts of the N-oxides and solvates of the N-oxides and salts thereof.

3. Compound having the systematic name ent-N-(2-amino-4,4-difluoro-2-methylbutyl)-8-[(2,6-difluorobenzyl)oxy]-2-methylimidazo[1,2-a]pyridine-3-carboxamide (enantiomer A) and the structural formula 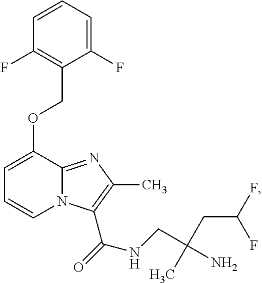 [SMILES: Cc1nc2c(OCc3c(F)cccc3F)cccn2c1C(=O)NCC(C)(N)CC(F)F]
and the N-oxides, salts, solvates, salts of the N-oxides and solvates of the N-oxides and salts thereof.

4. Compound having the systematic name ent-N-(2-amino-4,4-difluoro-2-methylbutyl)-8-[(2,6-difluorobenzyl)oxy]-2-methylimidazo[1,2-a]pyridine-3-carboxamide (enantiomer B) and the structural formula 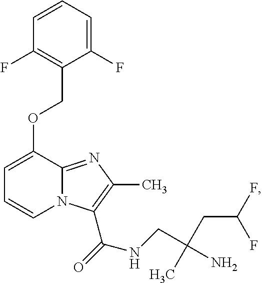 [SMILES: Cc1nc2c(OCc3c(F)cccc3F)cccn2c1C(=O)NCC(C)(N)CC(F)F]
and the N-oxides, salts, solvates, salts of the N-oxides and solvates of the N-oxides and salts thereof.

5. Compound having the systematic name ent-N-(2-amino-5,5,5-trifluoro-2-methylpentyl)-8-[(2,6-difluorobenzyl)oxy]-2-methylimidazo[1,2-a]pyridine-3-carboxamide (enantiomer A) and the structural formula 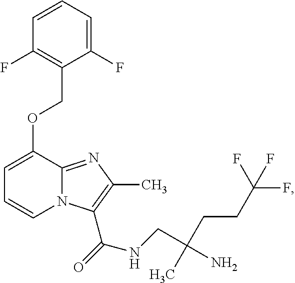 [SMILES: Cc1nc2c(OCc3c(F)cccc3F)cccn2c1C(=O)NCC(C)(N)CCC(F)(F)F]
and the N-oxides, salts, solvates, salts of the N-oxides and solvates of the N-oxides and salts thereof.

6. Compound having the systematic name ent-N-(2-amino-5,5,5-trifluoro-2-methylpentyl)-8-[(2,6-difluorobenzyl)oxy]-2-methylimidazo[1,2-a]pyridine-3-carboxamide (enantiomer B) and the structural formula 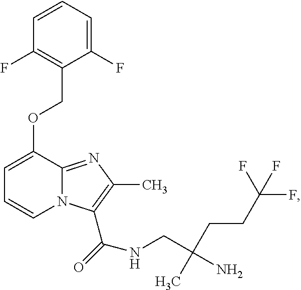 [SMILES: Cc1nc2c(OCc3c(F)cccc3F)cccn2c1C(=O)NCC(C)(N)CCC(F)(F)F]
and the N-oxides, salts, solvates, salts of the N-oxides and solvates of the N-oxides and salts thereof.

7. Compound having the systematic name ent-N-(2-amino-5,5,5-trifluoro-2-methylpentyl)-2-methyl-8-[(2,3,6-trifluorobenzyl)oxy]imidazo[1,2-a]pyridine-3-carboxamide (enantiomer B) and the structural formula 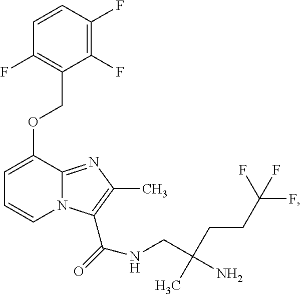 [SMILES: Cc1nc2c(OCc3c(F)ccc(F)c3F)cccn2c1C(=O)NCC(C)(N)CCC(F)(F)F]
and the N-oxides, salts, solvates, salts of the N-oxides and solvates of the N-oxides and salts thereof.

8. Process for preparing the compound of claim 1, comprising:
[A] reacting a compound of the formula (I) 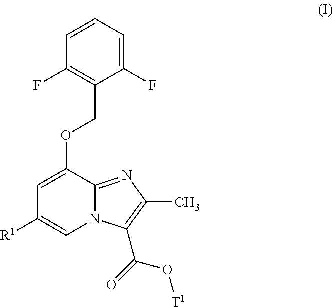
in which
R¹ represents hydrogen or chlorine,
T¹ represents (C₁-C₄)-alkyl or benzyl,
in an inert solvent in the presence of a suitable base or acid to give a carboxylic acid of the formula (II) 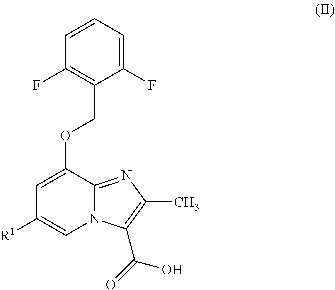
in which
R¹ represents hydrogen or chlorine,
and subsequently reacting the carboxylic acid of the formula (II) in an inert solvent under amide coupling conditions with an amine selected from the group consisting of 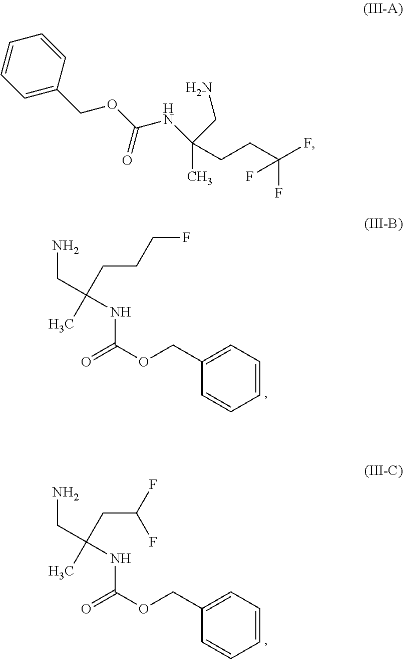
to give compounds of the formula (IV) 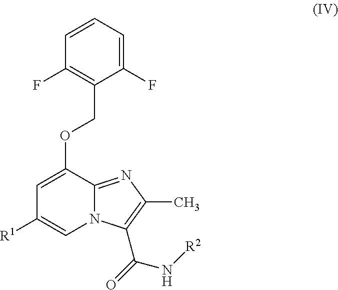
in which
R¹ represents hydrogen or chlorine,
and
R² represents (IV-A), (IV-B) or (IV-C) 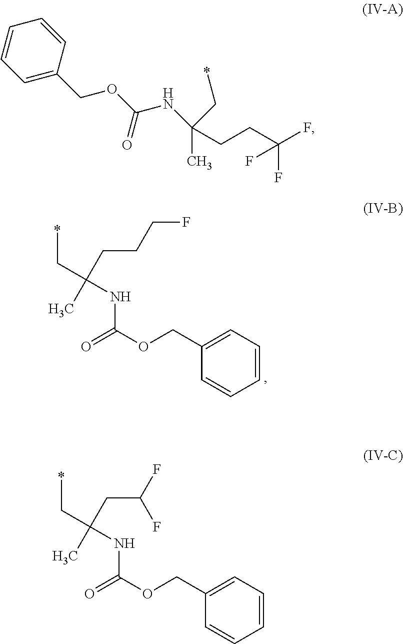 [SMILES: *CC(C)(CCC(F)(F)F)NC(=O)OCc1ccccc1 ; *CC(C)(CCCF)NC(=O)OCc1ccccc1 ; *CC(C)(CC(F)F)NC(=O)OCc1ccccc1]
where * represents the point of attachment to the nitrogen atom,
and, if R¹ represents chlorine,
hydrogenating these in an inert solvent in the presence of a suitable transition metal catalyst,
and optionally converting the resulting compounds with the appropriate (i) solvents and/or (ii) acids or bases to the solvates, salts and/or solvates of the salts thereof.

9. (canceled)

10. (canceled)

11. Medicament comprising the compound as defined in claim 1 in combination with an inert, nontoxic, pharmaceutically suitable excipient.

12. Medicament comprising the compound as defined in claim 1 in combination with a further active compound selected from the group consisting of organic nitrates, NO donors, cGMP-PDE inhibitors, antithrombotic agents, hypotensive agents and lipid metabolism modifiers.

13. (canceled)

14. Method for the treatment and/or prophylaxis of heart failure, angina pectoris, hypertension, pulmonary hypertension, ischaemias, vascular disorders, renal insufficiency, thromboembolic disorders and arteriosclerosis comprising administering an effective amount of at least one compound as defined in claim 1 to a human or animal in need thereof.

15. Method for treatment and/or prophylaxis of heart failure, angina pectoris, hypertension, pulmonary hypertension, ischaemias, vascular disorders, renal insufficiency, thromboembolic disorders and arteriosclerosis comprising administering an effective amount of the medicament of claim 11 to a human or animal in need thereof.

16. Method for treatment and/or prophylaxis of heart failure, angina pectoris, hypertension, pulmonary hypertension, ischaemias, vascular disorders, renal insufficiency, thromboembolic disorders and arteriosclerosis comprising administering an effective amount of the medicament of claim 12 to a human or animal in need thereof.
